(19) United States
(12) Patent Application Publication
(10) Pub. No.: US 2022/0338452 A1
(43) Pub. Date: Oct. 27, 2022

(54) ORGAN REGENERATION METHOD UTILIZING BLASTOCYST COMPLEMENTATION

(71) Applicant: The University of Tokyo, Tokyo (JP)

(72) Inventors: Hiromitsu NAKAUCHI, Tokyo (JP); Toshihiro KOBAYASHI, Tokyo (JP); Younsu LEE, Tokyo (JP); Joichi USUI, Tokyo (JP); Tomoyuki YAMAGUCHI, Tokyo (JP); Sanae HAMANAKA, Tokyo (JP)

(21) Appl. No.: 17/497,356

(22) Filed: Oct. 8, 2021

Related U.S. Application Data
(63) Continuation of application No. 16/356,570, filed on Mar. 18, 2019, now abandoned.
Application No. 16/356,570 is a continuation of application No. 15/375,982, filed on Dec. 12, 2016, now abandoned.
Application No. 15/375,982 is a continuation of application No. 12/583,559, filed on Aug. 21, 2009, now abandoned.
Application No. 12/583,559 is a continuation-in-part of application No. PCT/JP2008/051129, filed on Jan. 25, 2008.

(30) Foreign Application Priority Data
Feb. 22, 2007 (JP) 2007-042041
Nov. 30, 2007 (JP) 2007-311786

(51) Int. Cl. A01K 67/027; A61L 27/38

(52) U.S. Cl. CPC A01K 67/0271; A61L 27/3834; A01K 2207/20

(57) ABSTRACT

An object of the present invention is to produce a mammalian organ having a complicated cellular composition composed of multiple kinds of cells, such as kidney, pancreas, thymus and hair, in the living body of a non-human animal. The inventors of the present invention applied the chimeric animal assay described above, to a novel solid organ production method. More specifically, the inventors has shown that a model mouse which is deficient of kidney, pancreas, thymus or hair due to the dysfunction of the metanephric mesenchyme that is differentiated into most of an adult kidney, is rescued by blastocyst complementation by the chimeric animal assay, and whereby a kidney, a pancreas, thymus or hair can be newly produced.

### BACKGROUND

### Technical Field

The present invention relates to a method for producing an organ derived from a mammalian cell in vivo, using a cell derived from the organ to be produced, which is obtained from the same mammalian species.

### Description of the Related Art

In discussing regenerative medicine that is practiced in the form of cell transplantation and organ transplantation, expectations for pluripotent stem cells are high. Embryonic stem cells (ES cells) derived from the inner cell mass of blastocyst stage fertilized eggs are pluripotential, and thus are widely used in the study of differentiation of various cells. Development of differentiation control methods of inducing differentiation of ES cells into specific cell lineages in vitro is a topic in the research of regenerative medicine.

In the study of in vitro differentiation using ES cells, ES cells are likely to differentiate into the mesoderm and the ectoderm, such as blood cells, blood vessels, cardiac muscles and nervous systems, in the early stage of embryonic development. However, a general tendency is known such that differentiation into organs directed by the formation of complicated tissue structures through intercellular interaction after the middle stage of embryonic development.

For example, metanephros, which is the adult kidney of mammals, develops from the intermediate mesoderm during the middle stage of embryonic development. Specifically, the development of kidney is initiated by the interaction between two components, namely, a mesenchymal cell and ureteric bud epithelium, and finally, the adult kidney is completed by the differentiation into multiple types of functional cells, which count as many as several dozen types that cannot be seen in other organs, and the constitution of complicated nephron structures centered around the glomeruli and uriniferous tubules, resulting from the differentiation. Considering the complexities of the development time and the development process of kidney, it can be easily conjectured that inducing a kidney from ES cells in vitro would be a very laborious and difficult work, and it is considered practically impossible. Furthermore, in organs such as the kidney, the identification of somatic stem cells is still not definitive, and the contribution of bone marrow cells to the reparation of injured kidneys, which was once vigorously studied, has been revealed to be insignificant.

When pluripotent ES cells are injected into the inner space of a blastocyst stage fertilized egg, the resulting individual forms a chimeric mouse. There has been previously reported a rescue experiment of T-cell and B-cell lineages by blastocyst complementation, to which this technique is applied, in a Rag-2 knockout mouse deficient in T-cell and B-cell lineages (Non-Patent Document 1). This chimeric mouse assay is used as an in vivo assay system for verifying the differentiation of the T-cell lineage, which cannot be provided by in vitro assay systems.

However, even if such a technique is found to be effective in a certain organ, it is difficult to predict whether the technique will also be effective in other organs, because of the difference in the function of the organs in a living body, for example, the difference in fatality resulting from the absence of the organs, and various factors affect the validity of the technique. In addition, the deficient gene of the organ deficiency model selected in this instance is also an important factor. This is because it is required to select transcription factors that are essential for the function of the deficient genes during the development process, particularly for the differentiation and maintenance of stem/precursor cells of each organ during the process of organ formation.

It is expected that when a model representing organ deficiency caused by the deficiency of a humoral factor or a secretion factor is to be used, only the factors released are complemented by the factors released from the ES cell-derived cells, and a chimeric state is adopted at the level of the organ.

Accordingly, selection of an appropriate model animal for an organ is the key factor in the present invention, and upon considering the application to other organs, it is considered problematic to use a model showing the same phenotype as that of the embodiments of the present invention with respect to other organs.
Non-Patent Document 1: Chen J., et al., Proc. Natl. Aca. Sci. USA, Vol. 90, pp. 4528-4532, 1993
Non-Patent Document 2: Nishinakamura, R. et al., Development, Vol. 128, pp. 3105-3115, 2001
Non-Patent Document 3: Offield, M. F., et al., Development, Vol. 122, pp. 983-995, 1996
Non-Patent Document 4: McMahon, A. P. and Bradley, A., Cell, Vol. 62, pp. 1073-1085, 1990
Non-Patent Document 5: Kimura, S., et al., Genes and Development, Vol. 10, pp. 60-69, 1996
Non-Patent Document 6: Celli, G., et al., EMBO J., Vol. 17, pp. 1642-655, 1998
Non-Patent Document 7: Takasato, M., et al., Mechanisms of Development, Vol. 121, pp. 547-557, 2004
Non-Patent Document 8: Mulnard, J. G., C. R. Acad. Sci. Paris. 276, 379-381 (1973)
Non-Patent Document 9: Stern, M. S., Nature. 243, 472-473 (1973)
Non-Patent Document 10: Tachi, S. & Tachi, C. Dev. Biol. 80, 18-27 (1980)
Non-Patent Document 11: Zeilmarker, G., Nature, 242, 115-116 (1973)
Non-Patent Document 12: Fehilly, C. B., et al., Nature, 307, 634-636 (1984)
Non-Patent Document 13: Bevis B. J. and Glick B. S., Nature Biotechnology Vol. 20, pp. 83-87, 2002
Non-Patent Document 14: Poueymirou W T, et al., Nature Biotechnol. 2007 January; 25(1): 91-9

### BRIEF SUMMARY

It is an object of the present invention to produce a mammalian organ having a complicated cellular constitution formed from multiple kinds of cells, such as kidney, pancreas, hair and thymus, in the living body of an animal, particularly, a non-human animal.

### Means for Solving the Problems

The inventors of the present invention have applied the above-described chimeric animal assay to a novel generation method for solid organs. More specifically, the inventors have showed that a kidney, a pancreas, hair and a thymus can be newly produced by applying the above-described chimeric animal assay, specifically, by rescuing a model animal (a sall1 knockout mouse, a nude mouse, or the like) deficient of kidney, pancreas, hair or thymus because of the functional abnormality in the metanephric mesenchyme, which is differentiated into the most parts of an adult kidney, in a mouse in which LacZ gene has been knocked in (also knocked out) into the Pdx1 gene locus, through blastocyst complementation.

The deficient gene of the organ deficiency model selected herein is also an important factor, and selecting transcription factors that are essential for the functions of the deficient gene during the development process, particularly for the differentiation and maintenance of stem/precursor cells of each organ during the process of organ formation, has been a key factor of the present invention.

However, it will be understood that as long as the method of the present invention is found to be applicable to a certain organ, appropriate modifications can be applied to that organ, based on previous successful cases. This is because when there is an appropriate defective animal, and when the same analysis method is applied using fluorescent-labeled ES cells, iPS cells or the like as described in the present specification, it becomes clear of whether the constructed organ is derived from the host or from the ES cells, iPS cells or the like, and the success or failure of organ construction can be decided.

When a model representing organ deficiency caused by the deficiency of a humoral factor or a secretion factor is to be used, it has been expected that only the released factors are complemented by the factors released from the cells derived from ES cells, iPS cells or the like, and a chimeric state is adopted at the level of the organ. However, this time, a working system was found for a kidney, a pancreas, hair and thymus. Accordingly, in regard to these particular organs, those ordinarily skilled in the art can make appropriate modifications of design based on the information provided in the present specification. When making such modification of design, the following may be taken into consideration.

Another key in the present invention is the selection of a model that is completely deficient in an organ. There are many animals, such as mouse, exhibiting hypoplasia of organ when a single gene is deleted, owing to the level and redundancy of the gene expression. However, even in the case of using those animals, cells derived from ES cells, iPS cells or the like develop in cooperation with the native cells, and thus it is expected that a chimeric state is adopted at the level of the organ. Accordingly, selection of the model animal is a key factor in the present invention. Upon considering the application to other organs, it has been conceived that it is difficult to use a model exhibiting the same phenotype as that of the present invention with respect to other organs. However, this time, a working system has been found for a kidney, a pancreas, hair and thymus. Accordingly, in regard to these particular organs, those ordinarily skilled in the art can make appropriate modifications of design based on the information provided in the present specification.

In this regard, Non-Patent Document 14 describes a method for producing a novel knockout mouse. According to the method, an attempt has been made to produce a mouse which is derived from ES cells, iPS cells or the like completely from the first generation, by increasing contribution to the injection into an embryo in a stage preceding the blastocyst stage using a laser, and thus integrate individuals are produced. Therefore, generation of organs cannot be attempted.

Specifically, the present invention provides a method for producing a target organ in the living body of a non-human mammal having an abnormality associated with a lack of development of the target organ in the development stage, the target organ being derived from an allogeneic and/or xenogeneic mammal that is an individual different from the non-human mammal, the method including:

a) preparing a cell derived from the allogeneic and/or xenogeneic mammal;

b) transplanting the cell into a blastocyst stage fertilized egg of the non-human mammal;

c) developing the fertilized egg in the womb of a non-human surrogate parent mammal to obtain a litter; and

d) obtaining the target organ from an individual of the litter.

Thus, it has been found that the problems described above can be solved.

According to the present invention, cells to be transplanted are prepared in accordance with the species of animal for the organ to be produced. For example, if it is desired to produce a human organ, human-derived cells are prepared, and if it is desired to produce an organ of a mammal other than human, the mammal-derived cells are prepared. The cells to be transplanted according to the present invention are preferably cells having an ability to differentiate into the organ to be produced (totipotent cells or pluripotent cells), but they are not limited thereto. As the totipotent cells or pluripotent cells, embryonic stem cells (ES cells), induced pluripotent stem cells (iPS cells), somatic stem cells, cells of zygote inner cell mass, early embryonic cells, and the like may be used, but the cells are not limited thereto. For example, if it is desired to produce a human organ, induced pluripotent stem cells, multipotent germline stem cells, and the like may be used. Preferably, ES cells, or iPS cells having an ability equivalent thereto (Nature. 2007 Jul. 19; 448(7151):313-7; Cell. 2006 Aug. 25; 126(4):663-76) can be used. The cell to be transplanted according to the present invention may be from any origin, such as human, pig, rat, mouse, cattle, sheep, goat, horse, dog, chimpanzee, gorilla, orangutan, monkey, marmoset or bonobo.

The organ to be generated by the method of the present invention may be any solid organ with a fixed shape, such as kidney, heart, pancreas, cerebellum, lung, thyroid gland, hair or thymus, but the organ is preferably a kidney, a pancreas, hair, or a thymus. These solid organs are generated in the bodies of the litter, by developing totipotent cells or pluripotent cells within an embryo that serves as a recipient. Since the totipotent cells or pluripotent cells can form all kinds of organs when made to develop in an embryo, there is no restriction on the type of solid organ that can be generated depending on the type of the totiponent cells or pluripotent cells to be used.

On the other hand, the present invention is characterized in that an organ derived only from the transplanted cells is formed in the living body of an individual of the litter derived from a non-human embryo that serves as a recipient, and thus it is not desirable to have a chimeric cell composition of the cells derived from the recipient non-human embryo and the cells to be transplanted. Therefore, as for the recipient non-human embryo, it is desirable to use an embryo derived from an animal having an abnormality associated with a lack of development of the organ to be produced during the development stage, and the baby born therefrom is deficient of that organ. As long as the animal is an animal developing such organ deficiency, a knockout animal having organ deficiency as a result of the deficiency of a specific gene, or a transgenic animal having organ deficiency as a result of incorporating a specific gene may be used.

For example, in the case of producing a kidney as the organ, embryos of a Sall1 knockout animal having an abnormality in which the development of kidney does not occur during the development stage (Non-Patent Document 2), or the like, may be used as a recipient non-human embryo. In the case of producing a pancreas as the organ, embryos of a Pdx-1 knockout animal having an abnormality in which the development of pancreas does not occur during the development stage (Non-Patent Document 3), may be used as the recipient non-human embryo. In the case of producing a cerebellum as the organ, embryos of a Wnt-1 (int-1) knockout animal having an abnormality in which the development of cerebellum does not occur during the development stage (Non-Patent Document 4), may be used as the recipient non-human embryo. In the case of producing a lung or a thyroid gland as the organ, embryos of a T/ebp knockout animal having an abnormality in which the development of lung or thyroid gland does not occur during the development stage (Non-Patent Document 5), may be used as the recipient non-human embryo. Furthermore, embryos of a dominant negative-type transgenic variant animal model (Non-Patent Document 6) which overexpresses the deletion of an intracellular domain of fibroblast growth factor (FGF) receptor (FGFR), which causes deficiency of multiple organs including kidney, lung, and the like, may also be used. Alternatively, nude mice may also be used in the generation of hair or thymus.

The non-human animal as the origin of a recipient embryo as used in the present invention, may be any animal other than human, such as pig, rat, mouse, cattle, sheep, goat, horse, dog, chimpanzee, gorilla, orangutan, monkey, marmoset or bonobo. It is preferable to collect the embryos from a non-human animal having a size of adult that is similar to that of the animal species of the organ to be produced.

On the other hand, the mammal as the origin of the cell that is transplanted into a recipient blastocyst stage fertilized egg in order to form the organ to be produced, may be either a human or a mammal other than human, for example, pig, rat, mouse, cattle, sheep, goat, horse, dog, chimpanzee, gorilla, orangutan, monkey, marmoset or bonobo.

The recipient embryo and the cell to be transplanted may be in a homologous relationship or in a heterologous relationship. In one embodiment, the cell may be from a rat, and the non-human mammal may be a mouse

The cell to be transplanted, prepared as described above, can be transplanted in the inner space of the recipient blastocyst stage fertilized egg, and a chimeric cell mixture of a blastocyst-derived inner cell and the cell to be transplanted may be formed in the inner space of the blastocyst stage fertilized egg.

The blastocyst stage fertilized egg having a cell transplanted thereinto as described above, is transplanted in the womb of a pseudo-pregnant or pregnant female animal of the species from which the blastocyst stage fertilized egg serving as the surrogate parent is derived. This blastocyst stage fertilized egg is developed within the surrogate womb to obtain a litter. Then, a target organ can be obtained from this litter, as a mammalian cell-derived target organ.

The present invention is also intended to include a mammal produced according to the method of the present invention. The reason for including such an animal is that only the target organ has target genomes, and such chimera type mammals were not present in the past.

The present invention also provides a use of a non-human mammal having an abnormality associated with a lack of development of a target organ in the development stage, for the generation of the target organ.

The present invention also provides a set for producing a target organ. This set includes cells derived from: A) a non-human mammal having an abnormality associated with a lack of development of the target organ in the development stage, and B) a cell derived from an allogeneic and/or xenogeneic mammal that is an individual different from the non-human mammal.

Therefore, these and other advantages of the present invention will become apparent as the following detailed description is read.

### Effects of the Invention

According to the method of the present invention, it was possible to form a certain organ derived from a mammalian cell, in the living body of an individual causing deficiency of the organ because the individual has an abnormality associated with a lack of development of the organ in the development stage. Particularly, the method of the present invention could be applied even to an organ having a complicated cellular constitution, such as kidney. When a kidney is formed, the formed kidney became a regenerated kidney in which nearly all of the metanephric mesenchyme-derived tissues, except for the ureteric bud, originated from the cell transplanted into the inner space of the blastocyst stage fertilized egg. In addition to the kidney, the pancreas, the thymus and the hair also became a regenerated pancreas, a regenerated thymus, and regenerated hair, respectively, originating from the cells transplanted into the inner space of the blastocyst stage fertilized egg.

### BRIEF DESCRIPTION OF THE SEVERAL VIEWS OF THE DRAWINGS

FIGS. 1A and 1B are sets of photographs showing kidney development in a normal individual (FIG. 1A), and kidney development in a Sall1 knockout mouse (Sall1(−/−)) (FIG. 1B). The upper side shows a macroscopic finding of the intraperitoneal cavity, and the lower side shows hematoxylin and eosin stained images of a median section slice of the renal part.

FIGS. 2A-2C are diagrams showing the means for performing genotype determination for a homozygote knockout individual (Sall1(−/−)), a heterozygote individual (Sall1(+/−)), and a wild type individual (Sall1(+/+)). FIG. 2A is a diagram showing the detection of the expression of a GFP gene that has been knocked into the Sall1 gene locus, by fluorescence detection; FIG. 2B is a diagram showing that GFP-positive cells and GFP-negative cells can be discriminated by sorting with a cell sorter, based on GFP fluorescence; and FIG. 2C is a diagram showing that for the Sall1(−/−) cells, Sall1(+/−) cells, and Sall1(+/+) cells, the genotype can be determined by a PCR method.

FIG. 3 shows a GFP fluorescence developed image of the intraperitoneal cavity of an individual of a litter on the first day (P1) after birth.

FIGS. 4A-4D show the respective macroscopic findings, GFP fluorescence images (GFP), DsRed fluorescence images (DsRed), and superimposed fluorescence images of GFP and DsRed (Merge), for a heterozygote individual (Sall1(+/−)) (FIG. 4A); a homozygote knockout chimeric individual (Sall1(−/−)), in which a pluripotent cell (ES cell) incorporated with DsRed gene was transplanted into the inner space of a blastocyst stage fertilized egg (FIG. 4B); a heterozygote chimeric individual (Sall1(+/−)), in which a pluripotent cell (ES cell) incorporated with DsRed gene was transplanted into the inner space of a blastocyst stage fertilized egg (FIG. 4C); and a wild type chimeric individual (Sall1(+/+)), in which a pluripotent cell (ES cell) incorporated with DsRed gene was transplanted into the inner space of a blastocyst stage fertilized egg (FIG. 4D).

FIGS. 5A-5C show the respective macroscopic findings, and superimposed fluorescence images of GFP and DsRed, for a heterozygote individual (Sall1(+/−)) (FIG. 5A); a homozygote knockout chimeric individual (Sall1(−/−)), in which a pluripotent cell (ES cell) incorporated with DsRed gene was transplanted into the inner space of a blastocyst stage fertilized egg (FIG. 5B); and a heterozygote chimeric individual (Sall1(+/−)), in which a pluripotent cell (ES cell) incorporated with DsRed gene was transplanted into the inner space of a blastocyst stage fertilized egg (FIG. 5C).

FIGS. 6A-6D show the results of cell sorting of brain cells and kidney cells, for a heterozygote individual (Sall1(+/−)) (FIG. 6A); a homozygote knockout chimeric individual (Sall1(−/−)), in which a pluripotent cell (ES cell) incorporated with DsRed gene was transplanted into the inner space of a blastocyst stage fertilized egg (FIG. 6B); a heterozygote chimeric individual (Sall1(+/−)), in which a pluripotent cell (ES cell) incorporated with DsRed gene was transplanted into the inner space of a blastocyst stage fertilized egg (FIG. 6C); and a wild type chimeric individual (Sall1(+/+)), in which a pluripotent cell (ES cell) incorporated with DsRed gene was transplanted into the inner space of a blastocyst stage fertilized egg (FIG. 6D). The horizontal axis represents the fluorescence intensity of GFP, and the vertical axis represents the fluorescence intensity of DsRed. A gel electrophoresis image showing the results of genotype determination of the cells obtained from the brain derived from the homozygote knockout chimeric individual (Sall1(−/−)) (FIG. 6B), is shown together.

FIG. 7 shows the histological analysis of the kidney obtained as a result of transplanting a pluripotent cell (ES cell) into the inner space of a blastocyst stage fertilized egg of the homozygote (Sall1(−/−)).

FIG. 8 shows a method for production of a knockout mouse through Pdx1-Lac-Z knock-in and blastocyst complementation. An ES cell labeled with an epidermal growth factor protein (EGFP) is injected, under a microscope, into an embryo obtained by breeding Pdx1 hetero individuals. FIG. 8 is a conceptual diagram showing that the obtained individual is theoretically a knockout individual at a probability of ¼ according to Mendelian inheritance, and if contribution of the ES cell could be made, construction of a completely ES cell-derived pancreas is possible. As also disclosed in Development 1996 March; 122(3):983-95., it is known that the presence of the pancreas is confirmed in wt/Pdx1-LacZ, and the pancreas is absent in Pdx1-LacZ/Pdx1-LacZ.

FIG. 9 shows the experimental result of generation of pancreas through blastocyst complementation. From the left side, the number of injected ova, the number of transplanted embryos, the number of litter, the hair color, and the number of chimera determined from EGFP fluorescence under a fluorescent microscope are shown in a table. Since this is a line in which generally the generation is still progressing, the reduction of the incidence rate is found to be more than usual. However, the chimera ratio of the obtained mouse was sufficient to conduct the experiment. The numbers inside circles represent the order of conducting this experiment.

FIGS. 10A-10C show an example of the mouse of the present invention having a pancreas produced by blastocyst complementation. The upper side shows a Pdx1-LacZ knock-in (knockout) mouse (homo), and the pancreas is not present (FIG. 10A). The middle side shows introduction of a GPFES cell into the blastocyst of a Pdx1-LacZ knock-in (knockout) mouse (hetero), and the pancreas is present and is very partially GPF-positive (FIG. 10B). The lower side shows introduction of a GPFES cell into the blastocyst of a Pdx1-LacZ knock-in (knockout) mouse (homo), and a pancreas derived from a GFP-positive ES cell can be seen (FIG. 10C).

FIG. 11 is a photograph showing a real example of hair growth from a nude mouse by BC (Example 3).

FIGS. 12A-12C show FACS analysis of peripheral blood. While CD4-positive, CD8-positive T-cells are present in the peripheral blood of a wild type mouse, they are not present in a nude mouse (since thymus is not present, the differentiation of matured T-cells is not induced). However, if normal ES cells marked with green fluorescent protein (GFP) are introduced into the blastocyst of the nude mouse (BC, blastocyst complementation), the differentiation of both of GFP-negative T-cells (derived from hematopoietic stem cells of nude mouse of a host) and GFP-positive T-cells (derived from ES cell) is induced, and thus, it is even functionally obvious that thymus is constructed by ES cells. B-cells exist even in the nude mouse, and there is no special change. GPF-positive B-cells are derived from the ES cells. From the upper side, a nude mouse (FIG. 12A), a wild type mouse (FIG. 12B), and a blastocyst chimeric mouse (FIG. 12C) are represented. From the left side within each of FIGS. 12A, 12B, and 12C, FACS analysis results of T-cells, CD8+ cells, CD4+ cells, B-cells are shown.

FIG. 13 is a photograph showing the thymus of a wild type mouse.

FIG. 14 is a photograph taken when fluorescence is illuminated (negative) to the thymus of a wild type mouse.

FIG. 15 is a photograph of a nude mouse (no thymus exists).

FIG. 16 is a photograph showing illumination of fluorescence to the mouse of FIG. 15.

FIG. 17 is a photograph showing complementation of blastocyst of a nude mouse with GFP-marked ES cell in Example 4 (the thymus exists).

FIG. 18 is a photograph taken when fluorescence is illuminated to the mouse of FIG. 17 (GFP-positive thymus exists).

FIG. 19 is a photograph taken when fluorescence is illuminated to thymus taken out from the mouse of FIG. 7.

FIG. 20A shows male Pdx1(−/−) mice (founder: Pdx1(−/−) mouse with a pancreas complemented with murine iPS cell), and female Pdx1(+/−) mouse has been cross bred and a fertilized egg has been obtained. This egg has been grown to a blastocyst stage, and the resultant blastocyst was microinjected under microscope with 10 rat iPS cells marked with EGFP. This was transplanted in the womb of a pseudo-pregnant female animal. This blastocyst stage fertilized egg is developed within the surrogate womb to obtain a litter by Cesarean section in the stage where pregnancy is completed. Upon observation of EGFP fluorescence under fluorescent stereoscopic microscope, it turned out that litter numbers #1, #2 and #3 are chimeric based on the EGFP expression on the body surface. Upon the Cesarean section, pancreas had uniform expression of EGFP observed in #1 and #2, however, the pancreas of #3 exhibited partial expression of EGFP, in a mosaic manner. #4 is a litter-mate as is #1-#3, but lacks fluorescence from EGFP, and its pancreas was deficient upon the Cesarean section, and thus it was a non-chimeric Pdx1(−/−) mouse. Further, the spleen was removed from these newborn animals and blood cells prepared therefrom were dyed with a monoclonal antibody against murine or rat CD45, and analyzed with a flow cytometer. As a result, in litter numbers #1-#3, rat CD45 positive cells were observed in addition to murine CD45 positive cells, and thus it was confirmed that these are heterologous chimera between mouse and rat containing cells derived from the host mouse and rat iPS cells. Furthermore, almost all cells in the rat CD45 positive cell fractions exhibited fluorescence of EGFP, and thus the rat CD45 positive cell are derived from rat iPS cells marked with EGFP.

FIG. 20B shows confirmation of Pdx1 gene type by PCR with host mice litter No. #1 to #3. In order to confirm gene type of the host mice, murine CD45 positive cells, which are encompassed by dotted lines in FIG. 10, were collected and genomic DNA was extracted therefrom and PCR was conducted using primers which allow distinction between Pdx1 mutant allele and wild-type allele. As a result, in #1 and #2, only bands corresponding to mutant type were observed, and in litter No. #3, both bands of mutant type and the wild-type were detected. Therefore, it is understood that the genotype of the host is Pdx1(−/−) in #1 and #2, and in the litter No. #3, it is Pdx1(+/−). From these results, the present inventors have succeeded in the generation of rat pancreas in an individual mouse by applying heterologous blastocyst complementation technology using rat iPS cell as a donor in mice No. #1 and #2, Pdx1(−/−) mice, which should not originally have generated pancreases.

DESCRIPTION OF SEQUENCE LISTING
(Description of sequence listing)
(SEQ ID NO: 1) primer 1 (wild type allele):
agctaaagctgccagagtgc
(SEQ ID NO: 2) primer 2 (common):
caacttgcgattgccataaa
(SEQ ID NO: 3) primer 3 (mutant allele):
gcgttggctacccgtgata
(SEQ ID NO: 4) nested PCR primer 1 (wild type
allele):
agaatgtcgcccgaggttg
(SEQ ID NO: 5) nested PCR primer 2 (common):
tacagcaagctaggagcac
(SEQ ID NO: 6) nested PCR primer 3 (mutant allele):
aagagcttggcggcgaatg
(SEQ ID NO: 7) forward primer of Example 2:
CAATGATGGCTCCAGGGTAA
(SEQ ID NO:8) reverse primer of Example 2:
TGACTTTCTGTGCTCAGAGG
(SEQ ID NO: 9) Forward Primer for detection of
cell derived from injected embryo (mutant and wild
type):
ATT GAG ATG AGA ACC GGC ATG
(SEQ ID NO: 10) Reverse Primer for detection of
cell derived from injected embryo (mutant):
TTC AAC ATG ACT GCC AGC TCC
(SEQ ID NO: 11) Reverse Primer for detection of
cell derived from injected embryo (wild type):
TGT GAG CGA GTA ACA ACC

### DETAILED DESCRIPTION

Hereinafter, the present invention will be described. It should be understood throughout the present specification that expression of a singular form includes the concept of their plurality unless otherwise mentioned. Accordingly, articles for a singular form (e. g., “a,” “an,” “the,” etc. in English) include the concept of their plurality unless specifically mentioned. It should also be understood that the terms as used herein have definitions typically used in the art unless otherwise mentioned. Thus, unless otherwise defined, all scientific and technical terms have the same meanings as those generally understood by those skilled in the art to which the present invention pertain. If there is contradiction, the present specification (including the definition) takes precedence.

### Molecular Biology

The terms “protein,” “polypeptide,” “oligopeptide” and “peptide” as used herein have the same meaning and refer to an amino acid polymer having any length. This polymer may be a straight-chained, branched or cyclic polymer. An amino acid may be a naturally occurring or non-naturally occurring amino acid, or a variant amino acid. The term may also include those assembled into a composite of a plurality of polypeptide chains. The term also includes naturally occurring or artificially modified amino acid polymers. Such modification includes, for example, disulfide bond formation, glycosylation, lipidation, acetylation, phosphorylation, or any other manipulation or modification (for example, conjugation with a labeling moiety). This definition encompasses a polypeptide containing at least one amino acid analog (for example, non-naturally occurring amino acid, etc.), a peptide-like compound (for example, peptoid), and other variants known in the art, for example.

As used herein, the term “amino acid” may refer to a naturally occurring or non-naturally occurring amino acid as long as it satisfies the purpose of the present invention.

As used herein, the term “nucleic acid” is used interchangeably with gene, cDNA, mRNA, oligonucleotide, and polynucleotide. A particular nucleic acid sequence also encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternative splicing of exons. Other polypeptides derived from the same nucleic acid by read-through transcription are also encompassed by this definition. Any products of a splicing reaction (including recombinant forms of the splice products), are included in this definition. Alternatively, allelic mutants may also be encompassed in this range.

The terms “polynucleotide,” “oligonucleotide,” and “nucleic acid” as used herein have the same meaning and refer to a nucleotide polymer having any length. This term also includes an “oligonucleotide derivative” or a “polynucleotide derivative.” An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, an oligonucleotide derivative in which ribose in an oligonucleotide is substituted with 2′-methoxyethoxy ribose, and the like. Unless otherwise indicated, particular nucleic acid sequences are also intended to encompass conservatively-modified variants thereof (for example degenerate codon substitutions) and complementary sequences as well as sequences explicitly indicated. Specifically, degenerate codon substitutions may be produced by generating sequences in which the third positions of one or more selected (or all) codons are substituted with mixed-bases and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

As used herein, the term “nucleotide” may be a naturally occurring or non-naturally occurring nucleotide.

As used herein, the term “search” indicates that a given nucleic acid sequence is utilized to find other nucleic acid base sequences having a specific function and/or property either electronically or biologically, or using other methods. Examples of the electronic search include, but are not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)), and the like. Examples of the biological search include, but are not limited to, stringent hybridization, a microarray (microarray assay) in which genomic DNA is attached to a nylon membrane or the like or a microarray (microarray assay) in which genomic DNA is attached to a glass plate, PCR and in situ hybridization, and the like. In the present specification, it is intended that a corresponding gene identified by the aforementioned electronic search or biological search should also be encompassed in the genes (for example, Sall1, Pdx-1, etc.) used in the present invention.

In the present specification, a nucleic acid sequence hybridizing with a specific gene sequence can be used if it has a function. As used herein, the term “stringent conditions for hybridization” refers to conditions that a complementary chain of a nucleotide strand having similarity or homology with respect to a target sequence hybridizes preferentially with the target sequence and a complementary chain of a nucleotide strand not having similarity or homology does not substantially hybridize. The term “complementary chain” of a given nucleic acid sequence indicates a nucleic acid sequence (for example, T to A, C to G) paired on the basis of a hydrogen bond between bases of a nucleic acid. The stringent conditions are sequence-dependent, and are different according to various circumstances. A longer sequence specifically hybridizes at higher temperature. Generally, the stringent conditions are selected to be about 5° C. lower than the thermal melting point (Tm) for a specific sequence at a defined ionic strength and pH. Tₘ is a temperature at which 50% of a nucleotide complementary to a target sequence hybridizes with the target sequence in an equilibrium state under a defined ionic strength, pH and nucleic acid concentration. The “stringent conditions” are sequence-dependent, and will vary depending on a variety of environmental parameters. General guidelines for nucleic acid hybridization are found in Tijssen (Tijssen (1993), Laboratory Techniques In Biochemistry And Molecular Biology—Hybridization With Nucleic Acid Probes Part I, Chapter 2, “Overview of principles of hybridization and the strategy of nucleic acid probe assay,” Elsevier, New York).

Typically, the stringent conditions are conditions in which a salt concentration is less than about 0.1 M Nat, and typically a concentration of about 0.01 to 1.0 M Na (or other salt), at pH 7.0 to 8.3, and a temperature is at least about 30° C. for short nucleotide sequences (for example, 10 to 50 nucleotides), and at least about 60° C. for long nucleotide sequences (for example, longer than 50 nucleotides). The stringent conditions also can be achieved by adding a destabilizing agent such as formamide. The stringent conditions according to the present specification may be hybridization performed in a buffer solution containing 50% formamide, 1 M NaCl and 1% SDS (37° C.), and washing with 0.1×SSC at 60° C.

The term “polynucleotide hybridized under stringent conditions” as used herein refers to a polynucleotide hybridized under well-known conditions that are commonly used in the art. Such a polynucleotide may be obtained by a Colony Hybridization method, a plaque hybridization method, a Southern blotting hybridization method or the like, using a polynucleotide selected from the polynucleotides of the present invention as a probe. Specifically, such a polynucleotide may be identified by hybridization using a filter, on which a DNA derived from a colony or a plaque is immobilized, in the presence of 0.7 to 1.0 M NaCl at 65° C., followed by washing the filter with SSC (Sall1-sodium citrate) solution having 0.1- to 2-fold concentration (SSC solution at a 1-fold concentration contains 150 mM sodium chloride and 15 mM sodium citrate) at 65° C. Hybridization may be conducted according to the method described in experimental manuals, such as Molecular Cloning, 2nd ed., Current Protocols in Molecular Biology, Supplement 1-38, DNA Cloning 1: Core Techniques, A Practical Approach, Second Edition, Oxford University Press (1995), and the like. Preferably, sequences hybridized under stringent conditions herein exclude those sequences containing an A sequence only or a T sequence only. The term “hybridizable polynucleotide” as used herein refers to a polynucleotide which can hybridize with another polynucleotide under the above-described hybridization conditions. Specific examples of the hybridizable polynucleotide include a polynucleotide having at least 60% homology with the base sequence of a DNA encoding a polypeptide having an amino acid sequence that is specifically shown in the present invention, preferably a polynucleotide having at least 80% homology or a polynucleotide having at least 90% homology, and more preferably a polynucleotide having at least 95% homology.

An amino acid can be denoted by either the generally known three-letter symbol, or the one-letter symbol recommended by the IUPAC-IUB Biochemical Nomenclature Commission. Likewise, a nucleotide can also be denoted by the generally-accepted one-letter code.

The term “homology” of a gene as used in the present specification refers to the extent of identity of two or more gene sequences with each other. Therefore, the higher the homology between two certain genes, the higher the identity or similarity between their sequences. Whether two genes have homology may be determined by comparing their sequences directly, or in the case of a nucleic acid, by a hybridization method under stringent conditions. When two gene sequences are directly compared with each other, the genes have homology if the DNA sequences of the gene sequences are typically at least 50% identical, preferably at least 70% identical, more preferably at least 80%, 90%, 95%, 96%, 97%, 98%, or 99% identical.

According to the present specification, comparisons of similarity, identity, and homology between amino acid sequences and base sequences are calculated using BLAST, which is a tool for sequence analysis, and using default parameters. A search for identity may be performed using, for example, BLAST 2.2.9 (published on May 12, 2004) of NCBI. The value of identity according to the present specification is usually provided as a value aligned using the above-mentioned BLAST under the default conditions. However, when a higher value is obtained as a result of a change in the parameters, the highest value will be designated as the value of identity. When identity is evaluated in multiple domains, the highest value among the resulting values is designated as the value of identity.

As used herein, the term “corresponding” gene refers to a gene in a certain species, which has, or is anticipated to have, an action similar to that of a predetermined gene in a species as a reference for comparison. If there is a plurality of genes having such an action, the term refers to a gene having the same evolutionary origin. Therefore, a gene corresponding to a given gene (for example, sall1) may be an orthologue of the given gene. Therefore, genes corresponding to human genes may be found in other animals (mouse, rat, pig, rabbit, guinea pig, cattle, sheep, and the like) as well. Such a corresponding gene may be identified using a technique that is well known in the art. Therefore, for example, a corresponding gene in a certain animal may be found by searching a sequence database of the animal (for example, mouse, rat, pig, rabbit, guinea pig, cattle, sheep, and the like), using the sequence of a gene that serves as the reference for the corresponding gene, as a query sequence.

As used herein, a “fragment” refers to a polypeptide or a polynucleotide having a sequence length ranging from 1 to n−1, with respect to a full-length polypeptide or polynucleotide (the length is n). The length of the fragment may be appropriately varied in accordance with the purpose, and for example, in the case of a polypeptide, the lower limit of the length of the fragment may be 3, 4, 5, 6, 7, 8, 9, 10, 15, 20, 25, 30, 40, 50 and more amino acids. Lengths that are represented by integers but are not specified herein (for example, 11 and the like) may also be appropriate as the lower limit. Further, in the case of a polynucleotide, the lower limit of the length of the fragment may be 5, 6, 7, 8, 9, 10, 15, 20, 25, 30, 40, 50, 75, 100 and more nucleotides. Lengths that are represented by integers but are not specified herein (for example, 11 and the like) may also be appropriate as the lower limit. According to the present specification, the lengths of a polypeptide and a polynucleotide may be represented by the numbers of amino acids or nucleic acids, respectively. However, the numbers mentioned above are not intended to be absolute, and the numbers as the upper or lower limit are intended to include some numbers above and below the subject number (or, for example, ±10%), as long as the same function is maintained. For the purpose of expressing such intention, the expression “about” may be attached before the number. However, it should be understood that in the present specification, the presence or absence of “about” does not affect the interpretation of the number. According to the present specification, a useful length of a fragment may be determined based on whether at least one function is maintained among the functions of the full-length protein which serves as the reference for the fragment.

According to the present specification, the term “variant” refers to a material such as a polypeptide or a polynucleotide having been partially modified as compared to the original substance. Examples of such variant include a substitution variant, an addition variant, a deletion variant, a truncation variant, an allelic mutant, and the like. The term “allele” refers to genetic variants that belong to a same genetic locus, but are discriminated from each other. Therefore, the term “allelic mutant” means a variant that is in the relationship of allele with respect to a certain gene. The term “homolog” refers to a sequence having homology (preferably at least 60% homology, more preferably at least 80%, at least 85%, at least 90%, or at least 95% homology) with a certain gene within a certain species at the amino acid or nucleotide level. The method of obtaining such a homolog is apparent from the description of the specification.

According to the present specification, in order to produce a functionally equivalent polypeptide, addition, deletion, or modification of amino acid can also be carried out in addition to substitution of amino acid. The substitution of amino acid means substituting an original peptide with one or more, for example, 1 to 10, preferably 1 to 5, and more preferably 1 to 3, amino acids. The addition of amino acid means adding one or more, for example, 1 to 10, preferably 1 to 5, and more preferably 1 to 3, amino acids to an original peptide chain. The deletion of amino acid means deletion of one or more, for example, 1 to 10, preferably 1 to 5, and more preferably 1 to 3, amino acids from an original peptide. The modification of amino acid includes amidation, carboxylation, sulfation, halogenation, alkylation, phosphorylation, hydroxylation, acylation (for example, acetylation) and the like, but is not limited thereto. An amino acid to be substituted or added may be a naturally occurring amino acid, a non-naturally occurring amino acid, or an amino acid analogue. A naturally occurring amino acid is preferable.

These nucleic acids may be obtained by a known PCR method and may also be chemically synthesized. To these methods, for example, a site-directed mutagenesis method and a hybridization method may be combined.

As used herein, the term “substitution, addition, and/or deletion” of a polypeptide or polynucleotide refers to substitution, addition, or removal of an amino acid or a substitute thereof, or a nucleotide or a substitute thereof, in an original polypeptide or polynucleotide, respectively. The techniques for these substitution, addition and/or deletion are known in the art, and examples of the techniques include a site-specific mutagenesis and the like. These changes in a reference nucleic acid or polypeptide may occur at the 5′-terminal or 3′-terminal of this nucleic acid, or may occur at the amino terminal site or the carboxy terminal site of the amino acid sequence representing this polypeptide, or may occur at any site between those terminal sites so that the changes are present individually between residues of the reference sequence, as long as a desired function (for example, deficiency of kidney, deficiency of pancreas, or the like) is maintained. The substitution, addition, or deletion may occur in any number of times as long as it is once or more, and such a change may occur many times, as long as a desired function (for example, deficiency of kidney, deficiency of pancreas, or the like) is maintained. For example, the number of such change may be one or several, and preferably, up to 20%, up to 15%, up to 10% or up to 5% of the total length, or 150 or less, 100 or less, 50 or less, 25 or less, or the like.

In order to describe embodiments of the present invention specifically, a method of producing a kidney derived from cells of a mammal other than human in the living body of a mouse will be described.

### Non-Human Animal

In order to produce a kidney derived from cells of a mammal other than human in the living body of an animal such as mouse, an animal such as mouse having an abnormality associated with a lack of development of kidney in the development stage, is prepared. According to one embodiment of the present invention, a Sall1 knockout mouse (Non-Patent Document 2) can be used as a mouse having an abnormality associated with a lack of development of kidney in the development stage. If this animal is a homozygote knockout genotype of Sall1(−/−), the animal is characterized in that only the development of kidney is absent, and individuals of a litter have no kidney.

This mouse has no kidney and cannot survive if the deficiency of Sall1 gene is in a homozygous state (Sall1(−/−)). Thus, the deficiency of Sall1 gene is maintained in a heterozygous state (Sall1(+/−)). Mice in such a heterozygous state are bred with each other (Sall1(+/−)×Sall1(+/−)), and fertilized eggs are collected from the womb. The fertilized eggs develop at a probability ratio of Sall1(+/+):Sall1(+/−):Sall1(−/−)=1:2:1, in terms of probability. According to the present invention, an embryo of Sall1(−/−), which develops at a probability of 25%, is used. However, it is difficult to determine the genotype in the stage of early embryo, and thus, it is practical to determine the genotype of the litter after birth and to use only those individuals having the desired genotype of Sall1(−/−) in the subsequent processes.

This knockout mouse may have Sall1 gene knocked out in the preparation stage and have the gene of a fluorescent protein for detection, or green fluorescent protein (GFP), knocked in into the Sall1 gene region in a expressible state (Non-Patent Document 7). When the regulatory region of this gene is activated by knocking-in such a fluorescent protein, expression of GFP occurs instead of Sall1, and the deficiency state of the Sall1 gene can be determined by fluorescence detection.

The relationship between a recipient embryo and a cell to be transplanted in the present invention may be a homologous relationship or a heterologous relationship. There have been hitherto a large number of reports on the preparation of a chimeric animal in such a heterologous relationship in the related art, and for example, blastular chimeric animals between closely related animal species, such as the preparation of a chimera between rat and mouse (Non-Patent Document 8; Non-Patent Document 9; Non-Patent Document 10; and Non-Patent Document 11), or the preparation of a chimera between goat and sheep (Non-Patent Document 12), have been actually reported. Therefore, in the case of preparing a kidney derived from cells of a mammal other than human in the living body of a mouse according to the present invention, a certain heterologous organ may be prepared in a recipient embryo, based on these conventionally known chimera creation methods (for example, a method of inserting cells to be transplanted into a recipient blastocyst (Non-Patent Document 12)).

As used herein, a “non-human mammal” refers to an animal from which a chimeric animal or embryo and the like are generated in conjunction with a cell to be transplanted.

As used herein, an “allogeneic and/or xenogeneic mammal” refers to an individual mammal which is different from a non-human mammal having an abnormality associated with a lack of development of a target organ in the development stage.

As used herein, a “non-human surrogate parent mammal” refers to an animal in which a fertilized egg developed by transplanting a cell derived from an allogeneic and/or xenogeneic mammal into a blastocyst stage fertilized egg of a non-human mammal is developed in the womb thereof (serving as a surrogate parent).

While the phrases “non-human mammal” and “non-human surrogate parent mammal” are sometimes referred to as a “non-human host mammal” or a “host”, it should be understood that the “non-human mammal” and “non-human surrogate parent mammal” are different from each other and such a difference should be apparent to those skilled in the art in the context of the present invention.

### Cell to be Transplanted

Next, in order to describe a cell to be transplanted by taking a kidney as an example, a mouse ES cell, a mouse iPS cell (see, for example, Okita K, et al., Generation of germline-competent induced pluripotent stem cells. Nature 448(7151) 313-7(2007)) or the like is prepared as the cell to be transplanted to produce a kidney derived from cells of a mammal other than human. This cell has a wild type genotype (Sall1(+/+)) with respect to Sall1 gene and has an ability to develop into all kinds of cells in the kidney.

This cell may incorporate, prior to transplantation, a fluorescent protein for specific detection in a state of being capable of expression. For example, as a fluorescent protein for such detection, the sequence of DsRed.T4 (Non-Patent Document 13), which is a DsRed genetic mutant, may be designed so to be expressed in the organs of the entire body under the control of a CAG promoter (cytomegalovirus enhancer and chicken actin gene promoter), and may be incorporated into ES cells by electroporation. By performing a fluorescent labeling on these cells for transplantation, it can be easily detected as to whether a produced organ is composed of transplanted cells only.

This mouse ES cell or the like is transplanted into the inner space of a blastocyst stage fertilized egg having the aforementioned genotype of Sall1 (−/−) to prepare a blastocyst stage fertilized egg having a chimeric inner cell mass, and this blastocyst stage fertilized egg having a chimeric inner cell mass is developed in a surrogate womb to obtain a litter. It is to be understood that as for the cell species to be used, the present invention can utilize not only the ES cells, but also pluripotent cells such as iPS and multipotent germ stem cells, as long as the cells are capable of following the above procedure. iPS cells and multipotent germ stem cells can be used. For example, in order to prepare an iPS cell, Okita K et al., Ibid., may be referred. In the case of an iPS cell line called Nanog-iPS, which was produced based on this document, since the iPS cell line is not marked, the cells cannot be distinguished from the embryos of the host when used in the production of chimera, and it cannot be discriminated whether the complementation of organ has been achieved. Therefore, in order to solve the problem, a fluorescent dye can be introduced into this Nanog-iPS cell line, thereby being capable of carrying out an experiment with the same protocol as the case of using the ES cell. If the cell such as described above is used, it is possible to produce an organ with the same protocol as the case of using the ES cell, and to clarify the origin.

### Formation of Kidney

The formation of a kidney can be investigated by performing macroscopic or microscopic morphological analysis, gene expression analysis and the like, using methods such as visual inspection, microscopic observation after staining, or observation using fluorescence.

For example, by performing visual inspection, the actual presence or absence of the organ, and features of the organ, such as the external appearance, can be investigated. Together with such a macroscopic morphological analysis, a tissue obtained after general tissue staining, such as hematoxylin-eosin staining, may be observed microscopically using a microscopy. Such microscopic observation allows investigations, even including concrete various cellular compositions within the kidney.

Furthermore, the gene expression analysis using fluorescence, such as emission of fluorescence according to the conditions, may also be performed. For example, the above-mentioned Sall1 gene knockout mouse is characterized in that the fluorescence intensity is lower when the deficiency of the Sall1 gene is in the homozygous state (Sall1(−/−)), compared to the case of fluorescence where the deficiency of the Sall1 gene is in a heterozygous state (Sall1(+/−)). This is because GFP fluorescence occurs from both alleles in the former case, whereas fluorescence occurs only in one allele in the latter case. Using such a characteristic, it is possible to conveniently examine which genotype the target organ or the cell constituting the target organ would have with respect to the Sall1 gene. In the case of an iPS cell or a multipotent germ stem cell, for example, an iPS cell line called Nanog-iPS, since the cells are not marked, the cells cannot be distinguished from the embryos of the host when used in the production of chimera, and it cannot be discriminated whether the complementation of organ has been achieved. Therefore, in order to solve the problem, a fluorescent dye can be introduced into this Nanog-iPS cell line, whereby it is possible to produce an organ with the same protocol as the case of using the ES cell, and to clarify the origin.

### Formation of Pancreas

The formation of a pancreas can be investigated by performing macroscopic or microscopic morphological analysis, gene expression analysis and the like, using methods such as visual inspection, microscopic observation after staining, or observation using fluorescence.

For example, by performing visual inspection, the actual presence or absence of the organ, and features of the organ, such as the external appearance, can be investigated. Together with such a macroscopic morphological analysis, a tissue obtained after general tissue staining, such as hematoxylin-eosin staining, may be observed microscopically using a microscopy. Such microscopic observation allows investigations, even including concrete various cellular compositions within the pancreas.

Furthermore, the gene expression analysis using fluorescence, such as emission of fluorescence according to the conditions, may also be performed. For example, the above-mentioned knockout mouse based on Pdx1-Lac-Z knock-in is characterized in that in a wild type (+/+) or heterozygous (+/−) individual, when a fluorescent-labeled ES cell is used, mottled fluorescence in a chimeric state is shown even though the contribution is found. On the other hand, in a homozygous (−/−) individual, uniform fluorescence is shown because the pancreas is constructed by a cell that is completely derived from ES cells. Using such a characteristic, it is possible to conveniently examine which genotype the target organ or the cell constituting the target organ would have with respect to the Pdx1 gene. iPS cells and multipotent germ stem cells can be used. For example, in order to prepare an iPS cell, Okita K et al., Ibid., may be referred. In the case of an iPS cell line called Nanog-iPS, which was produced based on this document, since the iPS cell line is not marked, the cells cannot be distinguished from the embryos of the host when used in the production of chimera, and it cannot be discriminated whether the complementation of organ has been achieved. Therefore, in order to solve the problem, a fluorescent dye can be introduced into this Nanog-iPS cell line, thereby being capable of carrying out an experiment with the same protocol as the case of using the ES cell. If the cell such as described above is used, it is possible to produce an organ with the same protocol as the case of using the ES cell, and to clarify the origin.

### Formation of Hair

The formation of hair can be investigated by performing macroscopic or microscopic morphological analysis, gene expression analysis and the like, using methods such as visual inspection or observation using fluorescence.

For example, by performing visual inspection, the actual presence or absence of hair, and features of hair, such as the external appearance, can be investigated. Together with such a macroscopic morphological analysis, a tissue obtained after general tissue staining, such as hematoxylin-eosin staining, may be observed microscopically using a microscopy. Such microscopic observation allows investigations, even including concrete various cellular compositions within the hair.

Furthermore, the gene expression analysis using fluorescence, such as emission of fluorescence according to the conditions, may also be performed. For example, in the case of the above-mentioned nude mouse, because of strong self-fluorescence of the hair, it is very difficult to determine whether the produced hair is derived from the nude mouse or from the ES cell with naked eye under a fluorescence microscope. However, the observation can also be performed by a means for observing the fluorescence appropriately. Using such a characteristic, it is possible to conveniently examine which genotype the target organ or the cell constituting the target organ would have. iPS cells and multipotent germ stem cells can be used. For example, in order to prepare an iPS cell, Okita K et al., Ibid., may be referred. In the case of an iPS cell line called Nanog-iPS, which was produced based on this document, since the iPS cell line is not marked, the cells cannot be distinguished from the embryos of the host when used in the production of chimera, and it cannot be discriminated whether the complementation of organ has been achieved. Therefore, in order to solve the problem, a fluorescent dye can be introduced into this Nanog-iPS cell line, thereby being capable of carrying out an experiment with the same protocol as the case of using the ES cell. If the cell such as described above is used, it is possible to produce an organ with the same protocol as the case of using the ES cell, and to clarify the origin.

### Formation of Thymus

The formation of thymus can be investigated by performing macroscopic or microscopic morphological analysis, gene expression analysis and the like, using methods such as visual inspection, microphotographs, FACS or observation using fluorescence.

For example, by performing visual inspection, the actual presence or absence of the organ, and features of the organ, such as the external appearance, can be investigated. Together with such a macroscopic morphological analysis, a tissue after general tissue staining, such as hematoxylin-eosin staining, may be observed microscopically using a microscopy. Such microscopic observation allows investigations, even including concrete various cellular compositions within the thymus.

Furthermore, the gene expression analysis using fluorescence, such as emission of fluorescence according to the conditions, may also be performed. For example, the above mentioned nude mouse does not conventionally have thymus, however, this does not affect the survival of the nude mouse. Accordingly, the nude mouse is born and survives naturally without the thymus. The nude mouse has a characteristic that as fluorescence-labeled ES cell is injected thereinto by blastocyst complementation, a large number of the nude mice in which the contribution of the ES cell is confirmed have the thymus showing fluorescence. Using such a characteristic, it is possible to conveniently examine which genotype the target organ or the cell constituting the target organ would have.

The target organ obtained according to the present invention has a characteristic that it is derived completely from the different individual mammal. In the conventional method, a chimera was regenerated. Although not intended to be bound by theory, it is conceived that this is probably because the transcription factor is necessary to the functions of the deficient genes during the development process, particularly to the differentiation and maintenance of the stem/precursor cells of each organ during the development process. iPS cells and multipotent germ stem cells can be used. For example, in order to prepare an iPS cell, Okita K et al., Ibid., may be referred. In the case of an iPS cell line called Nanog-iPS, which was produced based on this document, since the iPS cell line is not marked, the cells cannot be distinguished from the embryos of the host when used in the production of chimera, and it cannot be discriminated whether the complementation of organ has been achieved. Therefore, in order to solve the problem, a fluorescent dye can be introduced into this Nanog-iPS cell line, thereby being capable of carrying out an experiment with the same protocol as the case of using the ES cell. If the cell such as described above is used, it is possible to produce an organ with the same protocol as the case of using the ES cell, and to clarify the origin.

The present invention also provides mammals produced by the method of the present invention. The animal itself is also valuable as an invention because the animals having such a target organ could not be produced before. Although not intended to be bound by theory, it is conceived that the reason why such animals could not be produced till now is probably because the defected organ due to the gene deficiency was necessary for survival, and there was no way to rescue them.

Furthermore, the present invention also provides use of non-human mammals having an abnormality associated with a lack of development of a target organ in the development stage, for generation of the target organ. Using a cell for this use was not sufficiently discussed before. Accordingly, the mammal itself is also valuable as an invention. Although not intended to be bound by theory, it is conceived that the reason why such animals could not be produced till now is probably because the absent organ due to the gene deficiency was necessary for survival, and it was impossible to maintain a target individual to sexual maturity.

The present invention also provides a set for generation of a target organ. The set includes: 1) a non-human mammal having an abnormality associated with a lack of development of an organ in development stage, and B) a cell derived from a different mammal of the same kind as that of the target organ. It is conceived that such a set of an animal and a cell itself is also valuable as an invention because the set of an animal and a cell could not be used in the production of the target animal before. Although not intended to be bound by theory, it is conceived that the reason why the use of the set of an animal and a cell could not have been discussed before is probably because the absent organ due to the gene deficiency was necessary for survival, and it was impossible to maintain a male-female pair of the target individuals to sexual maturity, in order to allow the male-female pair to mate.

### Cases of Other Stem Cells

As stem cells other than the ES cells, for example, iPS cells and multipotent germ stem cells or the like may be used. For example, in order to produce iPS cells, Okita K et al. Ibid. may be referred. In the case of an iPS cell line called Nanog-iPS, which was produced based on this document, since the iPS cell line is not marked, the cells cannot be distinguished from the embryos of the host when used in the production of chimera, and it cannot be discriminated whether the complementation of organ has been achieved. Therefore, in order to solve the problem, a fluorescent dye can be introduced into this Nanog-iPS cell line, thereby being capable of carrying out an experiment with the same protocol as the case of using the ES cell. If the cell such as described above is used, it is possible to produce an organ with the same protocol as the case of using the ES cell, and to clarify the origin.

Points to Remember when Using Various Animals

The case of using animals other than a mouse can be performed by applying the manner described in the example of the present specification, while paying attention to the following points. For example, regarding the production of a chimera in other species of animals, specifically in a species other than mice, there are many reports of chimera into which inner cell mass originating from an embryo or the ES cell in an embryo is injected, rather than reports of the establishment of pluripotent stem cell having a chimera forming ability, (rat:(Mayer, J. R. Jr. & Fretz, H. I. The culture of preimplantation rat embryos and the prosuction of allophonic rats. J. Reprod. Fertil. 39, 1-10(1974)); cattle:(Brem, G. et al. Production of cattle chimerae through embryo microsurgery. Theriogenology. 23, 182(1985)); pig:(Kashiwazaki N et. al Production of chimeric pigs by the blastocyst injection method Vet. Rec. 130, 186-187(1992)). However, even though the chimera into which inner cell mass is injected is used, the method described in the present specification may be applied. By using such inner cell mass, it may be substantially possible to complement a defected organ of a defected animal. In other words, for example, the cell is cultivated till blastocyst in vitro, a portion of inner cell mass is physically separated from the obtained blastocyst, and then, it may be injected into the blastocyst. A chimeric embryo can be produced by agglutinating 8-cell or morulas in mid-course.

In the case of using the chimera in which inner cell mass is injected instead of a pluripotent stem cell such as an ES cell, it should be noted in the example of the present specification that the following points need to be altered or corrected in use. However, it is understood that these are techniques falling within the scope of a well-known technique in the art.

In the case of using the chimera into which inner cell mass is injected instead of a pluripotent stem cell such as an ES cell, since it is not a cell line as different from the ES cell, a process of producing a separate embryo (egg collection after natural crossbreeding, or artificial fertilization) is needed. Since such a protocol is disclosed in the above document (rat:(Mayer, J. R. Jr. & Fretz, H. I. The culture of preimplantation rat embryos and the prosuction of allophonic rats. J. Reprod. Fertil. 39, 1-10(1974)); cattle:(Brem, G. et al. Production of cattle chimerae through embryo microsurgery. Theriogenology. 23, 182(1985)); pig:(Kashiwazaki N et. al Production of chimeric pigs by the blastocyst injection method Vet. Rec. 130, 186-187(1992)), these documents, if necessary, are incorporated herein by reference.

### General Technique

The molecular biological method, the biochemical method, and the microbiological method used in the present specification are well known and commonly used in the art, and are disclosed in, for example, Sambrook J. et al. (1989). Molecular Cloning: A Laboratory Manual, Cold Spring Harbor and its 3rd Ed. (2001); Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999). PCR Applications: Protocols for Functional Genomics, Academic Press, separate-volume laboratory medicine ‘Experimental technique for gene transfer & expression analysis’ Yodosha, 1997, and so on. The parts (or all) related to the present specification are incorporated herein by reference.

A DNA synthesis technique and nucleic acid chemistry for producing an artificially synthesized gene are disclosed in, for example, Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues:A Practical Approac, IRL Press; Adams, R. L. et al. (1992). The Biochemistry of the Nucleic Acids, Chapman&Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (1996). Bioconjugate Techniques, Academic Press, and so on. The parts related to the present specification are incorporated herein by reference.

Reference documents cited in the present specification, such as science documents, patents, and patent applications, are incorporated herein by reference in their entirety to an extent that each of which is specifically described.

The preferred embodiments have been described for easy understanding of the present invention. Hereinafter, the present invention will be described based on examples, however, the above description and the following examples are provided only for illustrative purposes and are not provided for the purpose of limiting the present invention. Therefore, the scope of the present invention is not limited to the embodiments or examples specifically described in the present specification and is limited only by the claims.

### EXAMPLES

### Example 1

### Kidney Development in Kidney-Deficient Strain of Mouse

In the present Example, it was investigated whether kidney development would occur, by transplanting mouse ES cells as pluripotent cells into a knockout mouse that was characterized by kidney deficiency.

As the knockout mouse characterized by kidney deficiency, a Sall1 knockout mouse (donated by Professor Ryuichi Nishinakamura at Institute of Molecular Embryology and Genetics, Kumamoto University) was used. Sall1 gene is a gene of 3969 bp, encoding a protein having 1323 amino acid residues, and this gene is a mouse homolog of the anterior-posterior region-specific homeotic gene spalt(sal) of Drosophila, and has been suggested by a pronephric tubule induction test in African clawed frogs to be important in kidney development (Non-Patent Document 2, Asashima Lab in Tokyo University). It was reported that this Sall1 gene was expressed and localized in the kidney, as well as in the central nervous system, auditory vesicles, heart, limb buds and anus in the mouse (Non-Patent Document 2).

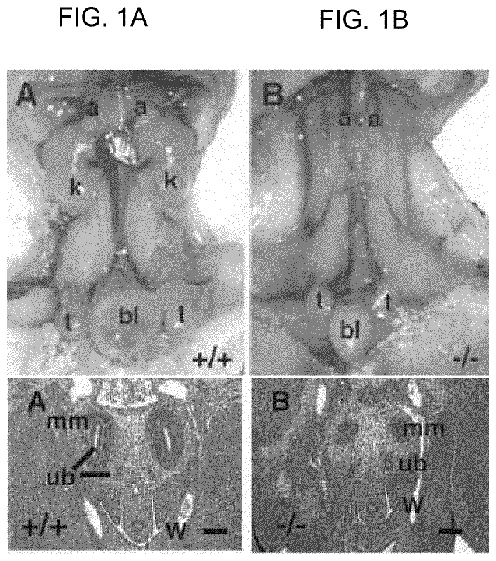

The knockout mouse of this Sall1 gene (backcrossed to C57BL/6 strain and analyzed) has exon 2 and subsequent parts in the Sall1 gene deleted, and thereby lacking all of the ten zinc finger domains present in the molecule. It is conceived that as a result of the deletion, interpolation of ureteric bud into the metanephric mesenchyme does not occur, and abnormality occurs in the initial stage of kidney formation (FIG. 1A: normal individual, FIG. 1B: Sall1 knockout mouse).

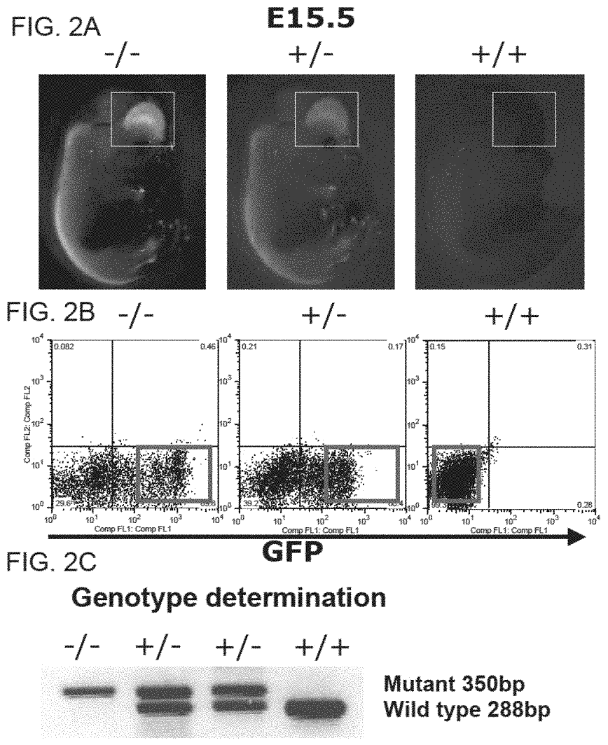

In the sall1 knockout mouse used in the experiment, GFP was knocked in as a marker in a sall1 gene locus, and thus the expression of sall1 gene was monitored using this detection system. As a result, in the sall1 knockout mouse (Sall1(−/−)), expression of GFP was confirmed in the fetal stage only, in limited organs such as central nervous system, kidney, four limbs, heart and paramesonephric duct. In the central nervous system, the sall1 gene was expressed, but any anatomical effect due to gene defect was not recognized. When the brain of a fetal mouse 15.5 days old was subjected to the detection of fluorescent development of GFP, it was found that intense fluorescence of GFP was emitted, in the order of the homozygote knockout individual (Sall1(−/−)), the heterozygote individual (Sall1(+/−)), and the wild type individual (Sall1(+/+)) (FIG. 2A). It was also found that when GFP-positive cells in this central nervous system were sorted by a cell sorter, the GFP-positive cells and GFP-negative cells could be clearly distinguished from each other (FIG. 2B).

Furthermore, PCR was performed using the full genome of the cells isolated by sorting as a template, and using

primer 1 (wild type allele):
(SEQ ID NO: 1)
agctaaagctgccagagtgc,
primer 2 (common):
(SEQ ID NO: 2)
caacttgcgattgccataaa,
primer 3 (mutant allele):
(SEQ ID NO: 3)
gcgttggctacccgtgata,
nested PCR primer 1 (wild type allele):
(SEQ ID NO: 4)
agaatgtcgcccgaggttg,
nested PCR primer 2 (common):
(SEQ ID NO: 5)
tacagcaagctaggagcac,
and
nested PCR primer 3 (mutant allele):
(SEQ ID NO: 6)
aagagcttggcggcgaatg,
to thus perform genotype determination.

The primer 1 was produced so as to hybridize with a nucleotide sequence corresponding to the gene defect part in a mutant allele among the genetic loci of Sall1, and thus hybridizes with a wild type allele only. The primer 2 was produced so as to hybridize with a nucleotide sequence that is present commonly in both wild type alleles and mutant alleles among the genetic loci of Sall1, and thus hybridizes with both a wild type allele and a mutant allele. The primer 3 was produced to hybridize with a nucleotide sequence in the GFP gene that has been inserted into the genetic loci of Sall1, and thus hybridizes with a mutant allele only.

Therefore, the sequence amplified by a combination of the primer 1 and the primer 2 is a portion of the genomic nucleotide sequence of the wild type genetic locus of Sall1, and the sequence amplified by a combination of the primer 2 and the primer 3 is a portion of the nucleotide sequence at the mutant type genetic locus of Sall1. As a result, the PCR product amplified by the combination of the primer 1 and the primer 2 is recognized as a wild type allele-derived nucleotide sequence having a size of 288 bp, while the PCR product amplified by the combination of the primer 2 and the primer 3 is recognized as a mutant allele-derived nucleotide sequence having a size of 350 bp.

In order to obtain more definite results, nested PCR primers were designed at the inner parts of the respective PCR products, and thus nested PCR was performed. The nested PCR primer 1 is a nucleotide sequence corresponding to the gene defect part in a mutant allele among the genetic loci of S all 1, and hybridizes only with the nucleotide sequences in the primer 2 binding site, rather than the primer 1 binding site, among wild type alleles. The nested PCR primer 2 is a nucleotide sequence that is present commonly in both wild type alleles and mutant alleles of the genetic loci of Sall1, and hybridizes with the nucleotide sequences in the primer 1 binding site or the primer 3 binding site, rather than the primer 2 binding site. The nested PCR primer 3 is a nucleotide sequence within the GFP gene that has been inserted into the genetic loci of Sall1, and hybridizes only with the nucleotide sequences in the primer 2 binding site, rather than the primer 3 binding site, among mutant alleles.

Therefore, the sequence amplified by a combination of the nested PCR primer 1 and the nested PCR primer 2 is a further portion of the portion of the genomic nucleotide sequence of the wild type genetic locus of Sall1 amplified by the combination of the primer 1 and the primer 2. The sequence amplified by a combination of the nested PCR primer 2 and the nested PCR primer 3 is a further portion of the portion of the nucleotide sequence at the mutant type genetic locus of Sall1 amplified by the combination of the primer 2 and the primer 3. As a result, the PCR product amplified by the combination of the nested PCR primer 1 and the nested PCR primer 2 is recognized as a wild type allele-derived nucleotide sequence having a size of 237 bp, and the PCR product amplified by the combination of the nested PCR primer 2 and the nested PCR primer 3 is recognized as a mutant allele-derived nucleotide sequence having a size of 302 bp.

When such genotype determination was performed, it was confirmed that genotype determination in a chimeric individual would be possible (FIG. 2C).

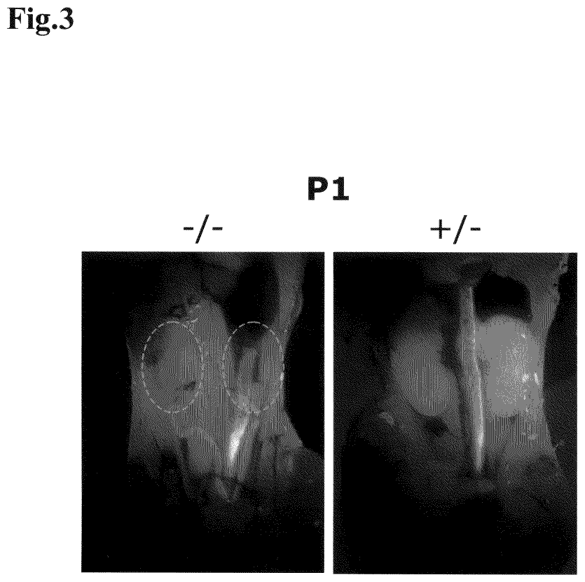

An investigation was made on the kidney formation in the individuals of a mouse litter one day after birth, which have been found to be homozygotes (Sall1(−/−)) or heterozygotes (Sall1(+/−)) according to the genotype determination, based on GFP expression. It was found that kidneys were formed in the heterozygotes (Sall1(+/−)), but kidneys were not at all formed in the homozygotes (Sall1(−/−)) (FIG. 3).

Male and female heterozygote individuals (Sall1-GFP(+/−)) of a sall1 gene knockout mouse in which the gene of green fluorescent protein (GFP) had been knocked in as a marker at a target genetic locus of sall1, were bred, and thus the blastocyst stage fertilized eggs were collected by a uterine reflux method. The genotype of the blastocyst stage fertilized eggs thus obtained was expected to appear at the ratio of homozygote (Sall1(−/−)):heterozygote (Sall1(+/−)):wild type (Sall1(+/+))=1:2:1.

Mouse ES cells marked with DsRed.T4 (Non-Patent Document 8) (129/Ola mouse-derived DsRed-EB3 cells, donated by Professor Niwa Hitoshi at RIKEN Center for Developmental Biology (Kobe)) were injected by microinjection into the collected blastocyst stage fertilized eggs at a rate of 15 cells per blastocyst, and the eggs were returned to a surrogate womb (ICR mouse, purchased from Japan SLC, Inc.).

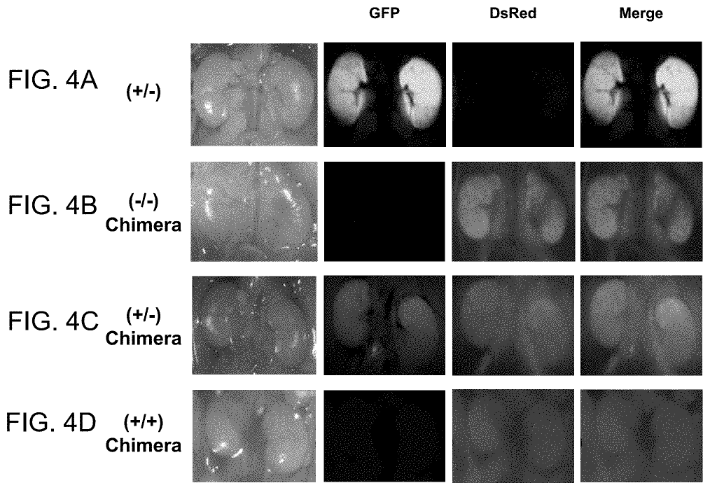

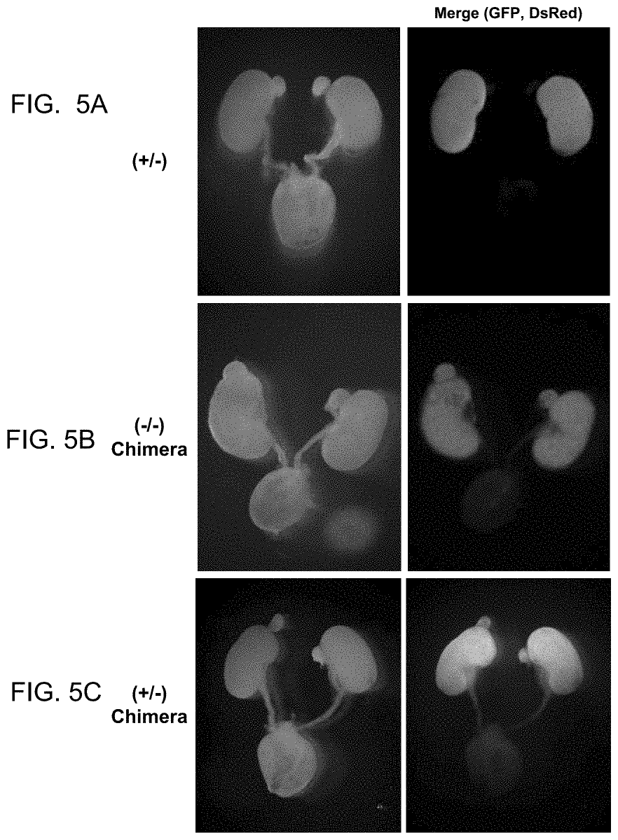

The neonatal chimeric individuals which could be confirmed to be homozygotes (Sall1(−/−)) by the genotype determination, had two normal-sized kidneys in the retroperitoneal area. When these formed kidneys were observed under a fluorescent stereoscopic microscope, the kidneys were strongly DsRed-positive (FIG. 4B, DsRed), and GFP-positive results were almost unverifiable (FIG. 4B, GFP and FIG. 5B). This indicates that in the homogyzotes (Sall1(−/−)), the kidneys were derived only from the mouse ES cells transplanted into the inner space of the blastocyst stage fertilized eggs. On the other hand, in the heterozygote (sall1(+/−)) individuals, since the kidneys were constituted of a chimera of the cells derived from the heterozygote (Sall1(+/−)) individuals and the cells derived from the transplanted ES cells, cellular images that were positive for both the fluorescence of GFP and the immunohistochemically derived fluorescence using an anti-DsRed antibody, were obtained (FIG. 4C and FIG. 5C).

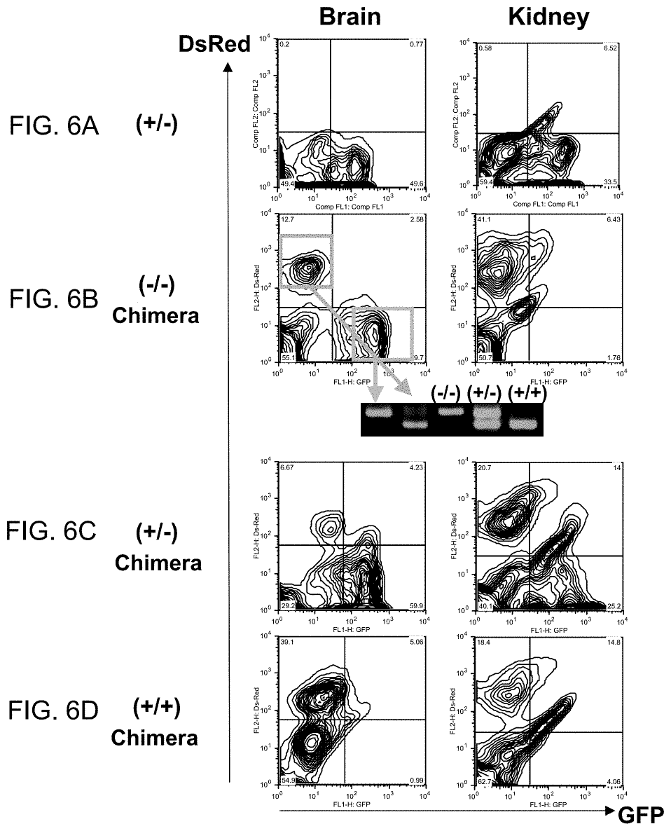

The brain and kidney cells of the homozygote (Sall1(−/−)) individuals thus obtained were sorted with a cell sorter based on GFP positive, and it was proved that in the brain cells, Sall1(−/−) cells (knockout mouse-derived cells) and Sall1(+/+) cells (ES cell-derived cells) constituted a chimera, while the kidney cells were constituted of Sall1(+/+) cells (ES cell-derived cells) only (FIG. 6B).

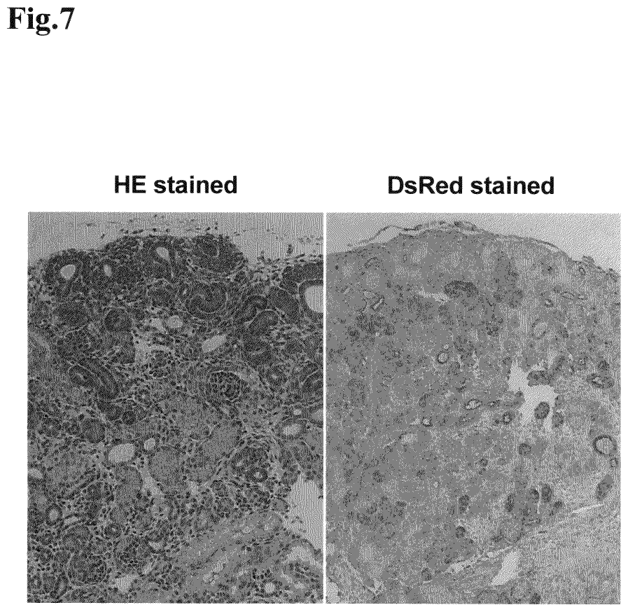

In the histological analysis of the kidneys obtained as a result of transplanting ES cells to the homozygote (Sall1(−/−)) blastocyst stage fertilized eggs, mature functional glomeruli containing erythrocytes in the loop cavity, and mature renal tubular structures could be observed (FIG. 7, HE-stained), and those mature cells were confirmed to be mostly DsRed-positive by an immunohistochemical analysis using an anti-DsRed antibody (FIG. 7, DsRed-stained).

From these results, it was confirmed that in the chimeric sall1 knockout mice (Sall1(−/−)) created by the method described above, the kidneys formed in the individuals of a litter were formed from the ES cells that had been transplanted into the inner space of the blastocyst stage fertilized eggs of the sall1 knockout mice (Sall1(−/−)).

### Example 2

### Pancreas Development in Pancreas-Deficient Strain of Mouse)

In the present Example, it was investigated whether pancreas development would occur, by transplanting mouse ES cells as pluripotent cells into a knockout mouse that was characterized by pancreas deficiency.

### Mouse Used

As a transgenic mouse characterized by pancreas deficiency, blastocysts derived from a mouse in which LacZ gene had been knocked in (also knocked out) at a Pdx1 gene locus (Pdx1-LacZ knock-in mouse), were used.

### Pdx1-LacZ Knock-In Mouse

In regard to the production of a construct, it can be produced, specifically based on the published article in Development 122, 983-995 (1996). In brief, the procedure is as follows. As for the arm of the homologous region, a product cloned from a λ clone including the Pdx1 region can be used. In the present Example, an arm donated by Professor Yoshiya Kawaguchi at the Laboratory of Surgical Oncology, Kyoto University Graduate School of Medicine, was used.

### Technique of Transgenic Knock-In: Pdx1-LacZ Knock-In Mouse

A clone obtained by introducing the construct described above into ES cells by electroporation, performing positive/negative selection, and then screening by Southern Blotting, was injected into blastocysts to thereby produce a chimeric mouse. Subsequently, a cell line developed into the germline is established, and the genetic background can be backcrossed into C57BL/6 strain to produce the mouse. In the present Example, a mouse donated by Professor Ryo Sumazaki at the University of Tsukuba was used, but the mouse can also be produced according to the protocol described above.

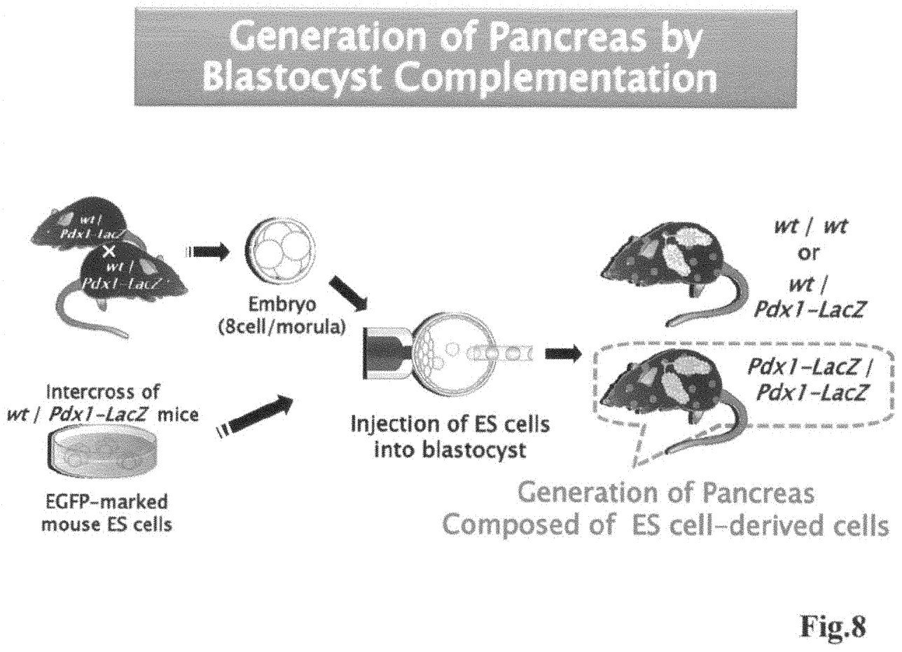

The scheme of the procedure is shown in FIG. 8.

### Breeding

Next, in the present Example, heterologous species of the mouse thus established were bred and used (FIG. 8). Since both animals of the knock-in mice described above were deficient of pancreas, the present Example was carried out under the concept of producing an ES cell-derived pancreas utilizing the vacancy.

In regard to the knock-in mice described above, since it was found that the mice could not survive homozygosity (died in about one week after birth), heterozygous mice were bred, and the embryos were recovered. Since it is understood that all of such matters do not constitute drawbacks which pose an obstacle in carrying out the present invention, the general versatility of the present invention is verified.

### Procedure for Maintenance of Mouse and Confirmation

ES cells were injected into blastocysts under a microscope using a micromanipulator. In this instance, a strain called G4.2, which was marked with EGFP, was used as the ES cells (donated by Professor Niwa Hitoshi at RIKEN CDB). The marked ES cells or the like which are equivalent to this strain may also be used. The embryos after the injection were transplanted into a surrogate womb, and thus a litter was obtained.

For the litter, if the litter is transgenic, the probability of the transgene being transferred to the next generation is ½, and if the litter is knock-in mice, the animals are homologous. In order to lower the probability to ¼, it is necessary to decide on which mouse is the desired “pancreas-deficient+ES cell-derived pancreas.” Therefore, the hit mouse was determined by collecting the cells of blood and tissues from both animals, isolating cells that were found to be EGFP-negative (not derived from ES cells, but cells derived from the injected embryos) by a flow cytometer, extracting the genomic DNA, and detecting the genotype by a PCR method.

The PCR primers used were as follows.

Forward:
(SEQ ID NO: 7)
CAATGATGGCTCCAGGGTAA
Reverse:
(SEQ ID NO: 8)
TGACTTTCTGTGCTCAGAGG

In regard to PCR, the process was performed in the same manner as in Example 1. The forward primer used was produced so as to hybridize with a nucleotide sequence corresponding to the Pdx1 promoter region, while the reverse primer was produced so as to hybridize with a nucleotide sequence of Hes1 cDNA (an mRNA whose Accession Number is NM_008235). Since such a Pdx1 promoter and Hes1 cDNA existing in the neighborhood cannot occur in wild type mice, it is possible to detect a transgene efficiently by PCR using these primers.

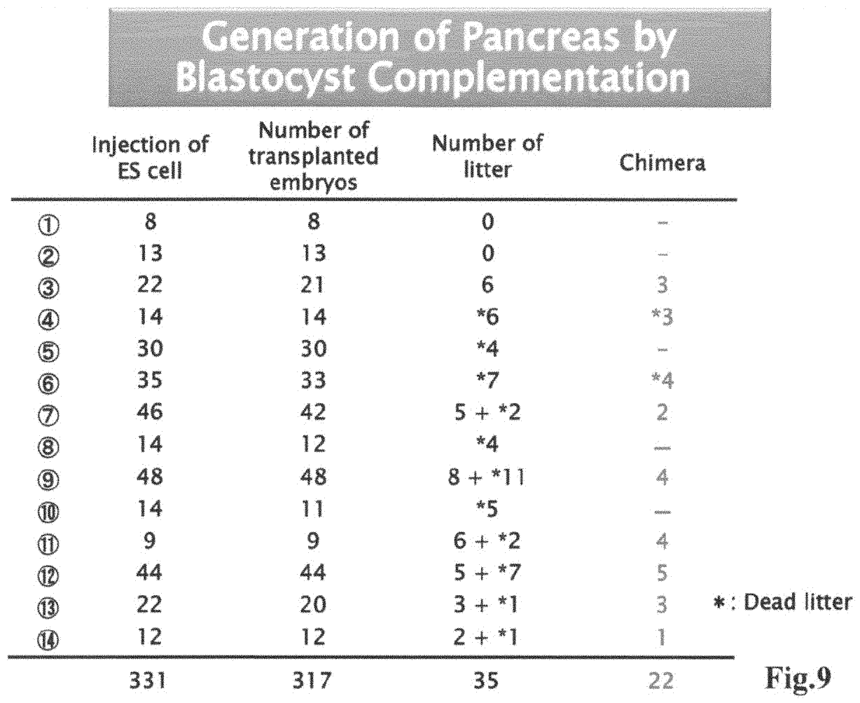

FIG. 9 depicts the results showing whether a pancreas has developed. FIG. 9 shows the results of Pdx1 knockout. The results show how efficiently a litter and a chimeric individual may be obtained.

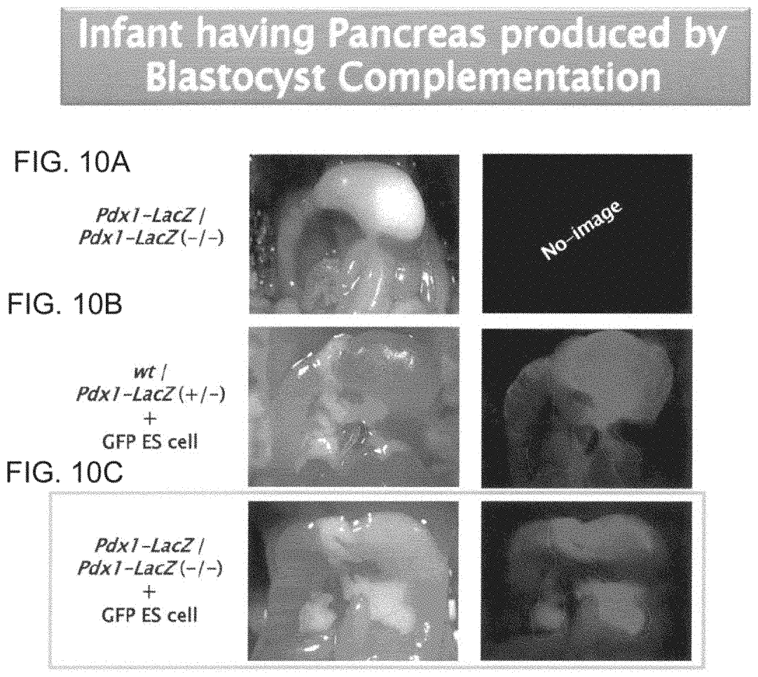

FIG. 10 shows an example of the mouse of the present invention having a pancreas produced by the blastocyst complementation. The upper side shows a Pdx1-LacZ knock-in (knock-out) mouse (homologous), and there is no pancreas. The middle side shows GFPES cells transferred into the blastocysts of a Pdx1-LacZ knock-in (knock-out) mouse (hetero), and a pancreas is present, and is very partially GFP-positive. The lower side shows GPFES cells transferred into the blastocysts of a Pdx1-LacZ knock-in (knock-out) mouse (homo), and a GFP-positive, ES cell-derived pancreas can be seen.

From the above, it was demonstrated that a pancreas could be produced according to the method of the present invention.

### Example 3

### Hair Growth in Hair-Deficient Strain of Mouse

In regard to the hair, it was investigated whether hair growth would occur, by using nude mouse-derived blastocysts, and transplanting mouse ES cells as pluripotent stem cells.

### Mouse Used

The mouse used was a nude mouse, and was purchased from Japan SLC, Inc. The nude mouse used was a sturdy nude mouse having good breeding efficiency, which was produced when nu gene of BALB/c nude mouse was introduced into an inbred DDD/1 strain of mouse.

ES cells were introduced into the blastocysts under a microscope using a micromanipulator. In this instance, a strain called G4.2, which was marked with an epidermal growth factor protein (EGFP), was used as the ES cells (donated by Professor Niwa Hitoshi at RIKEN CDB). The marked ES cells or the like which are equivalent to this strain may also be used. The embryos after the injection were transplanted into a surrogate womb, and thus a litter was obtained.

A nude mouse is a spontaneous model, and since animals deficient of thymus and hair do not cause any impediment in the survival and propagation, breeding between nude mice is possible. Accordingly, the entire litter includes nude mice, and thus determination of genotype is not necessary. Therefore, the confirmation by detection with PCR as in the case of Example 2 is also unnecessary.

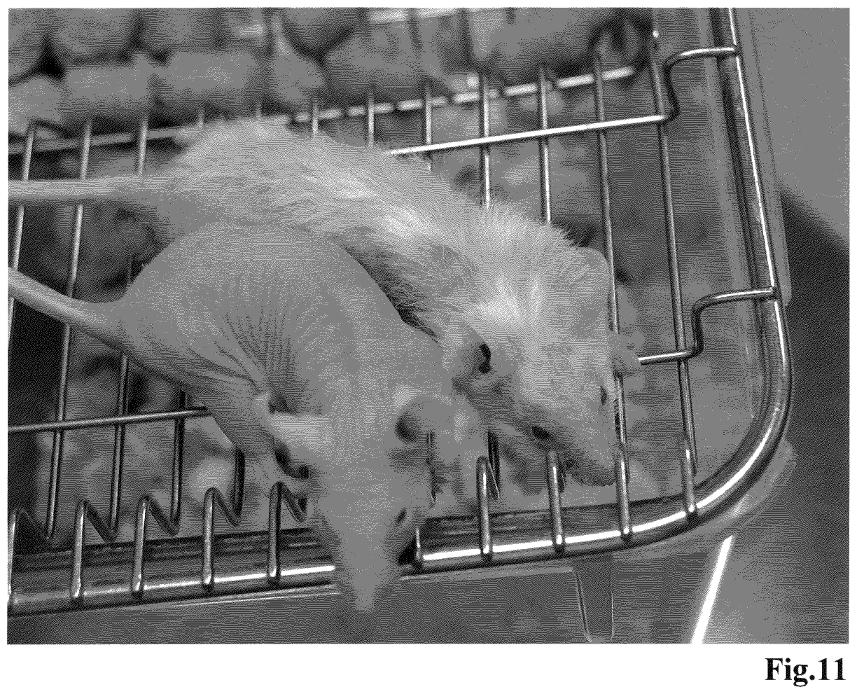

FIG. 11 depicts the results showing whether hair has developed. FIG. 11 shows a real example of a nude mouse developing hair according to the method of the present invention. From this result, the object that had developed was GFP-positive hair, and it was found that hair could regenerate.

In this case, the expression was weak. Thus, the same experiment was performed with B6 (can be purchased from RIKEN BRC)-derived ES cells, and whereby, it was confirmed that black hair grew out. As shown in FIG. 11, hair growth could be seen in the mouse on the right side that had been subjected to blastocyst complementation, as compared to the nude mouse on the left side.

### Conclusion

As discussed above, it was found that hair can be regenerated using the method of the present invention.

### Example 4

### Thymus Development in Thymus-Deficient Strain of Mouse

In regard to the thymus, it was investigated whether thymus development would occur, by using nude mouse-derived blastocysts, and transplanting mouse ES cells as pluripotent stem cells.

### Mouse Used

The mouse used was a nude mouse, and was purchased from Japan SLC, Inc. The nude mouse used was a sturdy nude mouse having good breeding efficiency, which was produced when nu gene of BALB/c nude mouse was introduced into an inbred DDD/1 strain of mouse.

### Procedure for Maintenance of Mouse and Confirmation

ES cells were injected into blastocysts under a microscope using a micromanipulator. In this instance, a strain called G4.2, which was marked with EGFP, was used as the ES cells (donated by Professor Niwa Hitoshi at RIKEN CDB). The marked ES cells or the like which are equivalent to this strain may also be used. The embryos after the injection were transplanted into a surrogate womb, and thus a litter was obtained. In the present Example, a nude mouse was used as described in Example 3, and thus confirmation by PCR was unnecessary.

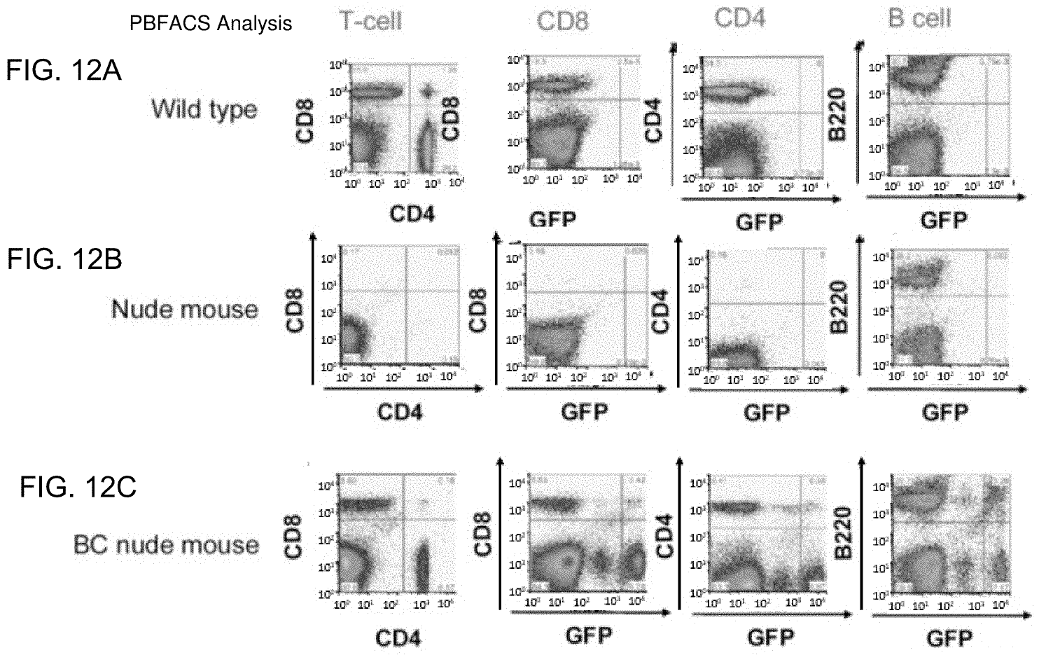

FIG. 12 depicts the results showing whether thymus has developed. Although CD4-positive and CD8-positive T cells were present in the peripheral blood of wild type mouse, the T cells were not present in the nude mouse (since thymus is not present, mature T cells are not induced to differentiate). However, when GFP-marked normal ES cells were introduced into the blastocysts of nude mouse (BC, blastocyst complementation), both of the GFP-negative T-cells (derived from hematopoietic cells of the host nude mouse) and the GFP-positive T-cells (derived from the ES cells) were induced to differentiate. Thus, it is obvious even from a functional viewpoint that thymus has been established by the ES cells. The B cells were also present in the nude mouse, without any particular changes. The GPF-positive B cells were derived from the ES cells. From the results of FIG. 12, as the newly established thymus properly functions, the immature T-cells that were originally present were induced to differentiate into CD4- and CD8-positive mature T-cells, and these could be detected in the peripheral blood. Therefore, it is understood that the T-cells show a chimera ratio corresponding to the proportion of contribution from the ES cells, as in the case of the B-cells.

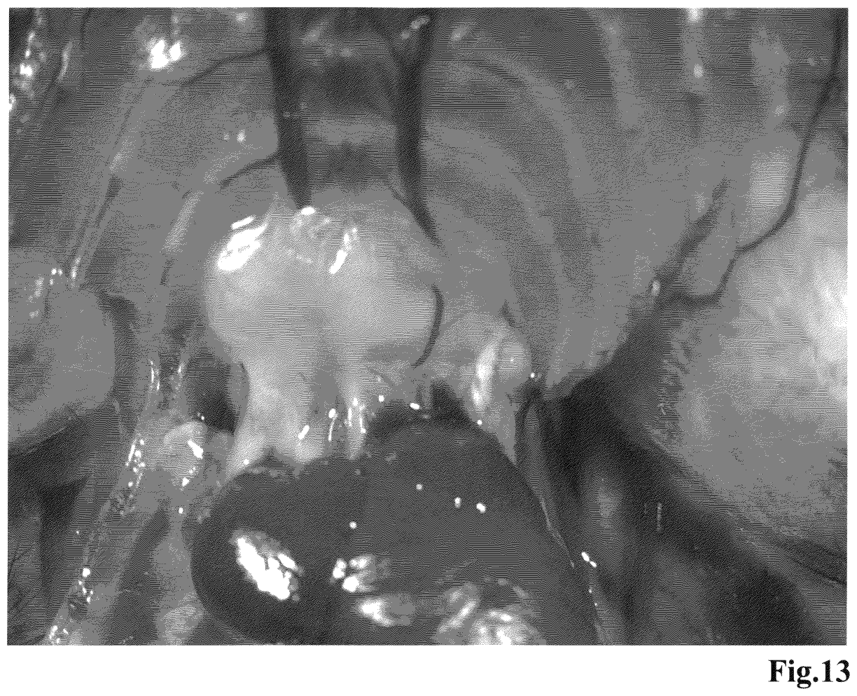

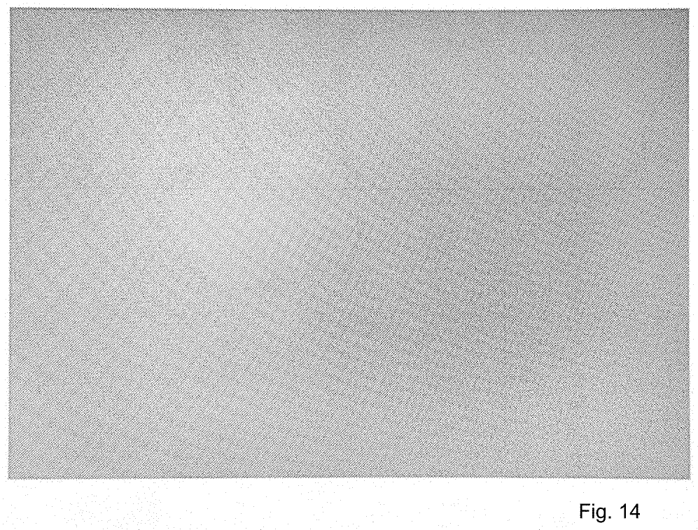

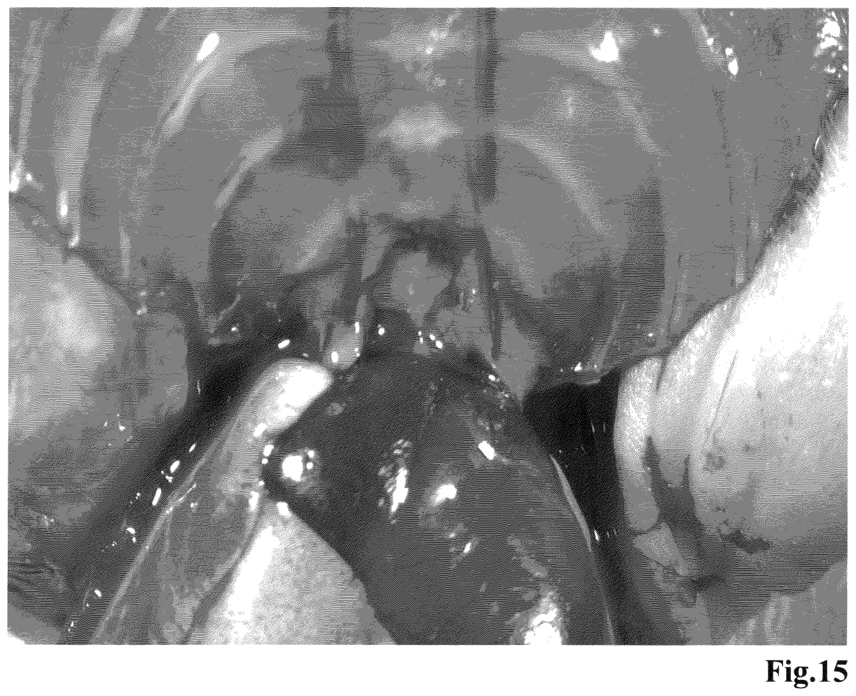

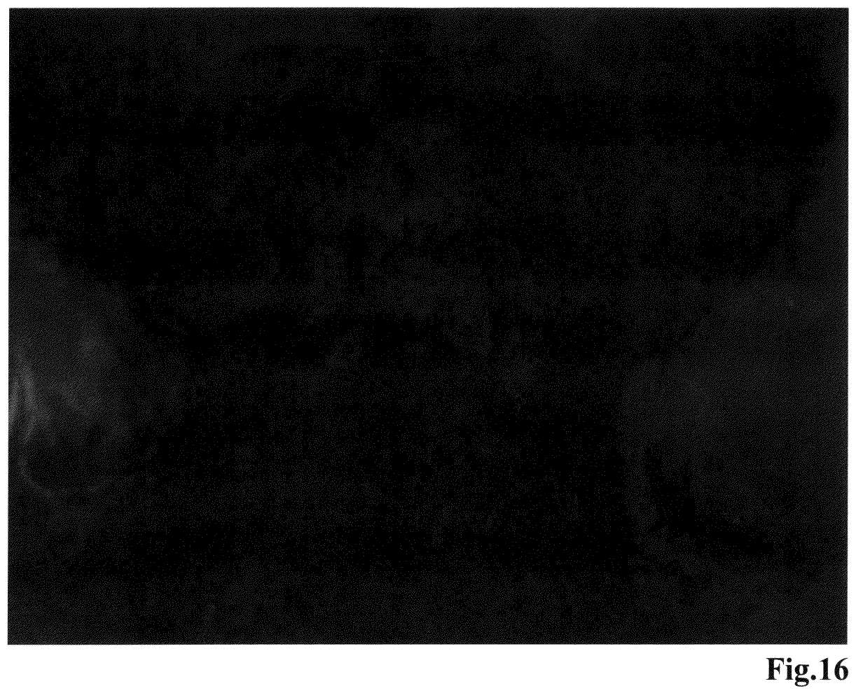

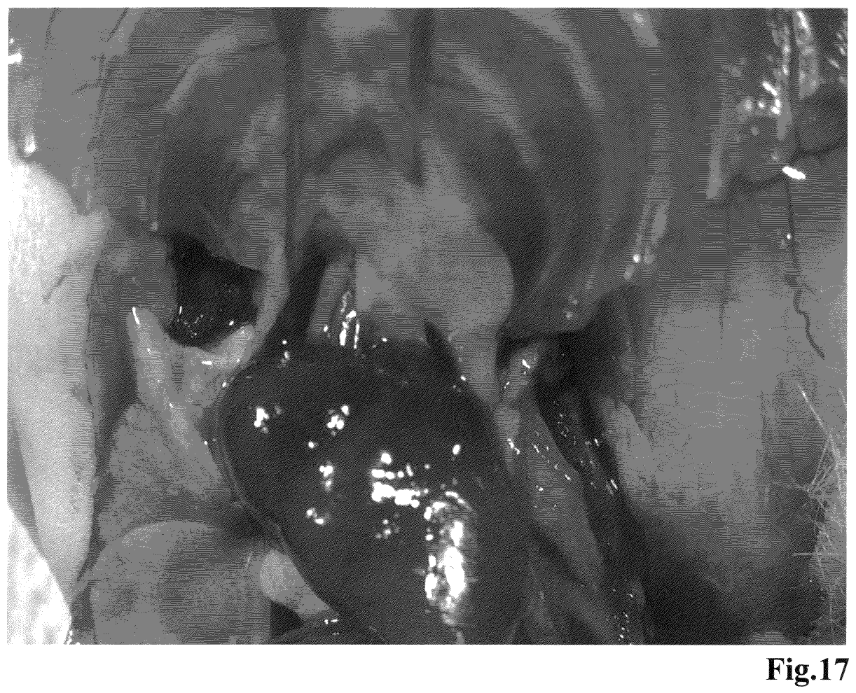

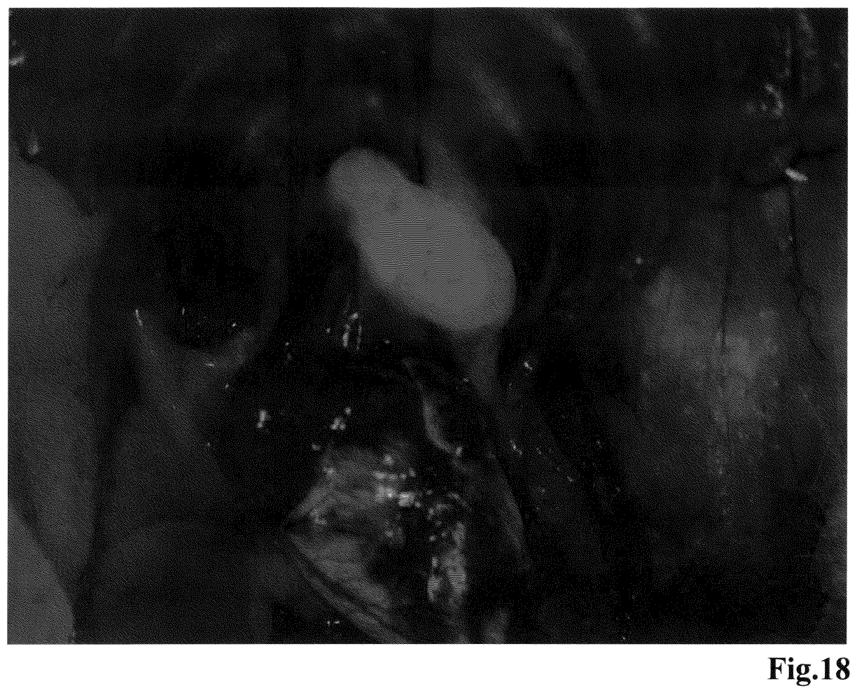

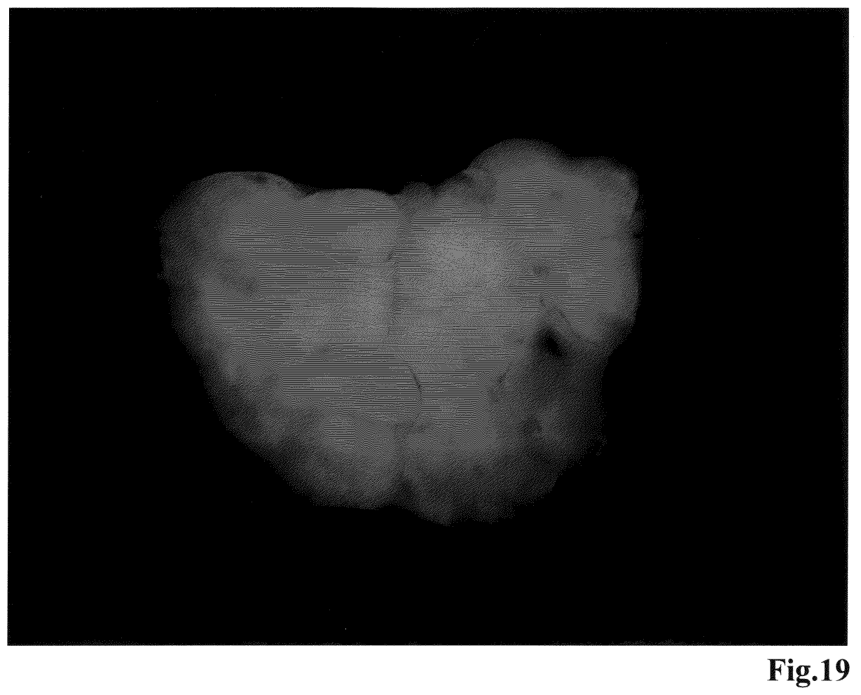

The photographs showing the development of thymus in the mice of the present invention, such as a nude mouse, a wild type mouse and a chimera, are shown in FIG. 13 to FIG. 19. FIGS. 13 and 14 show the photographs of the thymus of a wild type mouse, one showing the normal state and the other showing the fluorescence-illumination state (negative). FIGS. 15 and 16 show the photographs of the thymus of a nude mouse, one showing the normal state and the other showing the fluorescence-illumination state (no thymus). FIGS. 17 and 18 show the photographs of the thymus of a chimeric mouse carefully produced as described above, one showing the normal state and the other showing the fluorescence-illumination state (positive). FIG. 19 shows a photograph of the thymus extracted from this chimeric mouse, and illuminated of fluorescence. As shown, the thymus exhibited fluorescence, and it was proved that the tissue was derived from the ES cells.

### Conclusion

As discussed above, it was found that the thymus can be regenerated using the method of the present invention.

### Example 5

### Example of Using iPS Cells

An experiment is performed to confirm whether other pluripotent stem cells can be used in stead of the ES cells used in Examples 1 to 4. The induced type pluripotent stem cell, also known as iPS cell, is a cell successfully developed for the first time in the world by Professor Shinya Yamanaka at the Institute for Frontier Medical Sciences of Kyoto University, and its general versatility is attracting the public interest.

When cells as described above are used, it is possible to produce organs with the same protocol as that described in Examples 1 to 4, which was used in the case of using ES cells, and to clarify the origin.

For example, in the case of pancreas as described in Example 2, the process can be carried out as follows.

### Generation of Kidney

It is investigated whether kidney development occurs, by using the iPS cells produced as described above as pluripotent cells, and transplanting the iPS cells into the knockout mouse characterized by kidney deficiency used in Example 1.

As the knockout mouse characterized by kidney deficiency, the Sall1 knockout mouse described in Example 1 is used.

In the sall1 knockout mouse used in the experiment, GFP was knocked in as a maker at the genetic locus of sall1, and using this detection system, the expression of sall1 gene is monitored. As a result, in the sall1 knockout mouse (Sall1(−/−)), expression of huKO could be confirmed in the fetal stage only, in limited organs such as central nervous system, kidney, four limbs, heart and paramesonephric duct. The fluorescent color development of GFP was detected, and strong emission of the fluorescence of GFP could be confirmed, in the order of homozygote knockout individual (Sall1(−/−)), heterozygote individual (Sall1(+/−)), and wild type individual (Sall1(+/+)). Also, by sorting these GFP-positive cells of the central nervous system with a cell sorter, it can be confirmed that the GFP-positive cells and GFP-negative cells can be clearly distinguished from each other.

Further, the genotype determination can be carried out by performing PCR using the primers of SEQ ID NO:1 to 6 used in Example 1, and using the full genome of the cells isolated by sorting, as a template.

In order to obtain more definite results, nested PCR primers can be designed in the inner part of the respective PCR products, and nested PCR can also be performed as described in Example 1. By carrying out such genotype determination, it can be confirmed that it is possible to achieve genotype determination in chimeric individuals.

An investigation was made on the kidney formation in the individuals of a mouse litter one day after birth, which have been found to be homozygotes (Sall1(−/−)) or heterozygotes (Sall1(+/−)) in the above-described genotype determination, based on GFP expression. It can be confirmed that kidneys are formed in the heterozygotes (Sall1(+/−)), while kidneys are not formed at all in the homozygotes (Sall1(−/−)).

Male and female heterozygote individuals (Sall1-GFP(+/−)) of a sall1 gene knockout mouse in which the gene of green fluorescent protein (GFP) had been knocked in as a marker at a target genetic locus of sall1, were bred, and the blastocyst stage fertilized eggs were collected by a uterine reflux method. The genotype of the blastocyst stage fertilized eggs thus obtained is expected to appear at the ratio of homozygote (Sall1(−/−)):heterozygote (Sall1(+/−)):wild type (Sall1(+/+))=1:2:1.

The huKO marking iPS cells produced as described above are injected by microinjection into the collected blastocyst stage fertilized eggs at a rate of 15 cells per blastocyst, and the eggs are returned to a surrogate womb (ICR mouse, purchased from Japan SLC, Inc.).

It can be confirmed that the neonatal chimeric individuals which could be confirmed to be homozygotes (Sall1(−/−)) by the genotype determination, has two normal-sized kidneys present in the retroperitoneal area. When these formed kidneys are observed under a fluorescent stereoscopic microscope, it can be confirmed that the kidneys are strongly huKO-positive, and any GFP-positive finding is almost unverifiable. This indicates that in the homogyzote (Sall1(−/−)), the kidneys are derived only from the mouse iPS cells transplanted into the inner space of the blastocyst stage fertilized eggs. On the other hand, in the heterozygote (sall1(+/−)) individuals, since the kidneys are constituted of a chimera of the cells derived from the heterozygote (Sall1(+/−)) individuals and the cells derived from the transplanted iPS cells, cellular images that are positive for both the fluorescence of GFP and the immunohistochemically derived fluorescence using an anti-huKO antibody, can be obtained.

The brain and kidney cells of the homozygote (Sall1(−/−)) individuals thus obtained were sorted with a cell sorter based on GFP positive, and it is proved that in the brain cells, Sall1(−/−) cells (knockout mouse-derived cells) and Sall1(+/+) cells (ES cell-derived cells) constituted a chimera, while the kidney cells are constituted of Sall1(+/+) cells (iPS cell-derived cells) only.

In the histological analysis of the kidneys obtained as a result of transplanting iPS cells to the homozygote (Sall1(−/−)) blastocyst stage fertilized eggs, mature functional glomeruli containing erythrocytes in the loop cavity and mature renal tubular structures can be observed (HE-stained), and those mature cells can be confirmed to be mostly huKO-positive by an immunohistochemical analysis using an anti-huKO antibody (huKO-stained).

From these results, it can be confirmed that in the chimeric sall1 knockout mouse (Sall1(−/−)) created by the method described above, the kidneys formed in the individuals of a litter were formed from the ES cells that had been transplanted into the inner space of the blastocyst stage fertilized eggs of the sall1 knockout mouse (Sall1(−/−)).

### Generation of Pancreas

Technique of Transgenic Knock-In: Pdx1-LacZ Knock-In Mouse

A clone obtained by introducing the construct described above into the labeled iPS cells by electroporation, performing positive/negative selection, and then screening by Southern Blotting, was injected into blastocysts to thereby produce a chimeric mouse. Subsequently, a cell line developed into the germline can be established, and thereby the genetic background can be backcrossed into C57BL/6 strain to produce the mouse.

The procedure can be carried out according to the procedure depicted in FIG. 8.

### Breeding

Next, in the present Example, heterologous species of the mouse thus established can be bred and used. Since both animals of the knock-in mice described above were deficient of pancreas, the present Example is carried out under the concept of producing a labeled iPS cell-derived pancreas utilizing the vacancy.

In regard to the knock-in mice described above, since it was found that the mice could not survive homozygosity (died in about one week after birth), heterozygous mice were bred, and the embryos were recovered. Since it is understood that all of such matters do not constitute drawbacks which pose an obstacle in carrying out the present invention, the general versatility of the present invention is verified.

### Procedure for Maintenance of Mouse and Confirmation

The labeled iPS cells are injected into blastocysts under a microscope using a micromanipulator. In this instance, a strain marked with the aforementioned huKO is used for the labeled iPS cells. The marked iPS cells or the like which are equivalent to this strain may also be used. The embryos after the injection are transplanted into a surrogate womb, and thus a litter is obtained.

For the litter, if the litter is transgenic, the probability of the transgene being transferred to the next generation is ½, and if the litter is knock-in mice, the animals are homozygous. In order to lower the probability to ¼, it is necessary to decide on which mouse is the desired “pancreas-deficient+iPS cell-derived pancreas.” Therefore, the hit mouse can be determined by collecting the cells of blood and tissues from both animals, isolating cells that are found to be EGFP-negative (not derived from labeled iPS cells, but cells derived from the injected embryos) by a flow cytometer, extracting the genomic DNA, and detecting the genotype by a PCR method.

The PCR primers used are as follows.

Forward:
(SEQ ID NO: 7)
CAATGATGGCTCCAGGGTAA
Reverse:
(SEQ ID NO: 8)
TGACTTTCTGTGCTCAGAGG

In regard to PCR, the process can be performed in the same manner as in Example 1. The forward primer used is produced so as to hybridize with a nucleotide sequence corresponding to the Pdx1 promoter region, while the reverse primer is produced so as to hybridize with a nucleotide sequence of Hes1 cDNA (an mRNA whose Accession Number is NM_008235). Since such a Pdx1 promoter and Hes1 cDNA existing in the neighborhood cannot occur in wild type mice, it is possible to detect a transgene efficiently by PCR using these primers.

In regard to whether a pancreas has developed, the development can be confirmed by visual inspection.

### Example 6

### Development of Hair or Thymus in Hair or Thymus Deficient Strain of Mouse

In regard to regenerated hair or thymus using iPS cells, it can be investigated whether development of hair or thymus occurs, by using nude mouse-derived blastocysts, and transplanting the iPS cells produced in Example 5 as pluripotent stem cells.

### Mouse Use

The mouse used was a nude mouse, and was purchased from Japan SLC, Inc. The nude mouse used is a sturdy nude mouse having good breeding efficiency, which is produced when nu gene of BALB/c nude mouse is introduced into an inbred DDD/1 strain of mouse.

iPS cells are injected into the blastocysts under a microscope using a micromanipulator. These iPS cells are marked as shown in Example 5. The marked iPS cells or the like which are equivalent thereto may also be used. The embryos after the injection are transplanted into a surrogate womb, and thus a litter can be obtained.

A nude mouse is a spontaneous model, and since animals deficient of a thymus and hair do not cause any impediment in the survival and propagation, breeding between nude mice is possible. Accordingly, the entire litter includes nude mice, and thus determination of genotype is not necessary. Therefore, the confirmation by detection with PCR as in the case of Example 2 is also unnecessary.

Whether hair or thymus has developed can be confirmed by visual inspection. From this result, the object that had developed is huKO-positive hair or thymus, and it can be confirmed that hair or thymus can regenerate.

### Example 7

### Example of Using Animal Other than Mouse

In the present Example, it is demonstrated that organs can be produced even in the case of using animals other than mice. In regard to species other than mice, there are many reports of chimera into which inner cell mass originating from an embryo or the ES cells in an embryo is injected, rather than the reports of the establishment of pluripotent stem cells having an ability to form a chimera. Thus, organ generation can be carried out using this information. In the case of rat, the same experiment can be carried out using the information described in Mayer, J. R. Jr. & Fretz, H. I. The culture of preimplantation rat embryos and the prosuction of allophonic rats. J. Reprod. Fertil. 39, 1-10 (1974). In the case of cattle, the same experiment can be carried out using the information described in Brem, G. et al. Production of cattle chimerae through embryo microsurgery. Theriogenology, 23, 182 (1985). In the case of pig, the same experiment can be carried out using the information described in Kashiwazaki N et al., Production of chimeric pigs by the blastocyst injection method Vet. Rec., 130, 186-187 (1992).

For example, in the present Example, hair or thymus can be produced using a nude rat (for example, available from Nippon Crea Co., Ltd.).

Even in the case of using a rat, a similar experiment can be carried out in a manner equivalent to Examples 3 and 4. However, since it is difficult to obtain ES cells, the rat cells can be cultured in vitro to blastocysts, the inner cell mass is physically partially separated from the obtained blastocysts, and the inner cell mass can be injected into the blastocysts. Eight-celled embryos or morulas can be aggregated in mid-course, and whereby a chimeric embryo can be produced.

Using the chimeric embryo thus obtained, the same experiment as that of Example 3 or 4 can be carried out.

Furthermore, a nude rat is a spontaneous model, and since animals deficient of thymus and hair do not cause any impediment in the survival and propagation, breeding between nude mice is possible. Accordingly, the entire litter includes nude mice, and thus determination of genotype is not necessary. Therefore, the confirmation by detection with PCR as in the case of Example 2 is also unnecessary.

Whether hair or thymus has developed can be confirmed by visual inspection. From this result, the object that had developed is huKO-positive hair or thymus, and it can be confirmed that hair or thymus can regenerate.

As discussed above, the present invention was illustrated using preferred embodiments of the present invention, but it is understood that the scope of the present invention should be construed only by the claims. It is understood that the patents, patent applications and articles cited in the present specification should be such that the disclosures thereof are incorporated into the present specification by reference, as with the disclosures themselves are specifically described in the present specification.

### Example 8: Blastocyst Complementation Between Heterologous Animals

In the subject Example, Pdx1 knocked out mice deficient of pancreases, it was investigated whether blastocyst complementation between heterologous animals would occur, by using Pdx1 knocked out mice deficient of a pancreas as a host animal, and rat iPS cell (EGFP+) generated by a method according to the above mentioned Preparation Example.

### A. Animals Used:

As in Example 1, a heterozygous individual (Pdx1(+/−)) of the Pdx1 gene knocked out mice, was used as a knocked out mouse deficient of a pancreas and a homozygous individual (Pdx1(−/−); founder) which is complemented with pancreas by murine iPS cells.

### B. Preparation of Rat iPS Cells:

1) Construction of Vectors for Preparation of Rat iPS Cells

TRE from pTRE-Tight (Clontech), Ubiquitin C promoter, tTA from pTet-on advance (Clontech) and pIRES2EGFP (Clontech) are incorporated into the Lentivirus vector CS-CDF-CG-PRE multicloning sites from 5′ end. Murine Oct4, Klf4 and Sox2 are ligated with F2A and T2A, respectively, and inserted between said TRE of the lentivirus vector and the Ubiquitin C promoter to produce the subject vector (LV-TRE-mOKS-Ubc-tTA-I2G).

2) Establishment of Rat iPS Cells

Wistar rat fetal fibroblast cells within five passages (E14.5) were placed on dish coated with 0.1% gelatin, and cultured in a DMEM supplemented with 15% Fetal calf serum, 1% penicillin/streptomycin/L-glutamin (SIGMA). The following day of the inoculation, LV-TRE-mOKS-Ubc-tTA-I2G vector was used to produce a lentivirus, which was added to the culture solution to achieve viral infection to the cells. Twenty four hours later, the culture medium was replaced, and was placed on MEF treated with mitomycin C, and cultured on DMEM containing 1 μg/ml doxicyclin, 1000U/ml rat LIF (Millipore), supplemented with 15% FCS, 1% penicillin/stretomycin/L-glutamin. The following day, the culture medium was replaced with serum-free N2B27 medium (GIBCO) supplemented with 1 μg/ml doxicyclin, 1000U/ml rat LIF (Millipore) on every other day, from Day 7, inhibitors (2i;3 mM CHIR99021(Axon), 1 mM PD0325901 (Stemgent), 3i;2i+2 mM SU5402(CalbioChem)) were added. Colonies having appeared on Day 10 or later were picked up, and re-placed on MEF feeder. riPS cells such established were transplanted to blastocyst of non-human host mammals by maintaining passages using trypsin-EDTA every three or four days.

### C. Heterologous Blastocyst Complementation

Male Pdx-1(−/−) mice and female Pdx-1(+/−) mice were cross bred and fertilized eggs were obtained by means of uterine perfusion method. The fertilized eggs thus obtained were advanced to blastocyst in vitro, and the above-mentioned rat iPS cells marked with EGFP were injected to the resultant blastocyst at 10 cells per blastocyst by means of microinjection under a microscope. This was transplanted to the womb of a pseudo-pregnant female animal (ICR mouse, obtained from Japan SLC, KK, Japan), and Cesarean section was conducted upon completion of pregnancy, and the resultant newborn litters were analyzed.

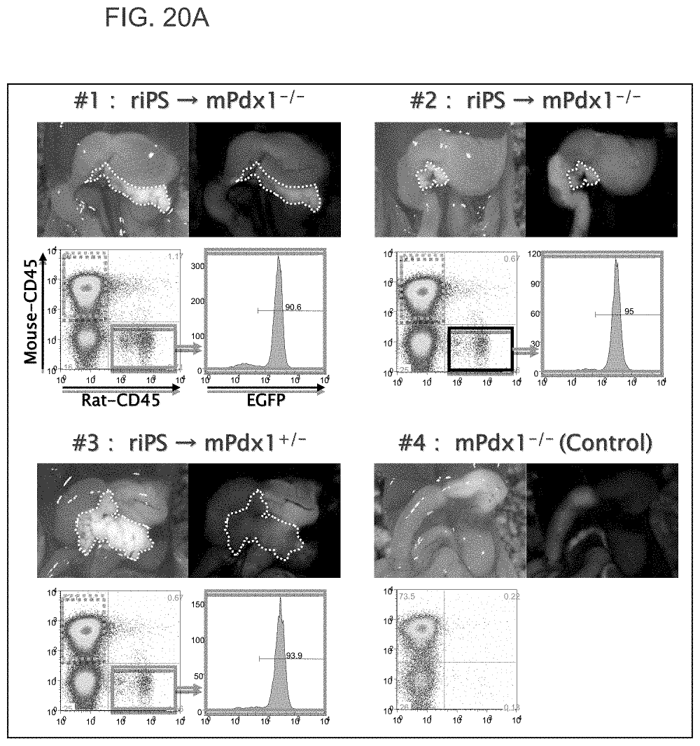

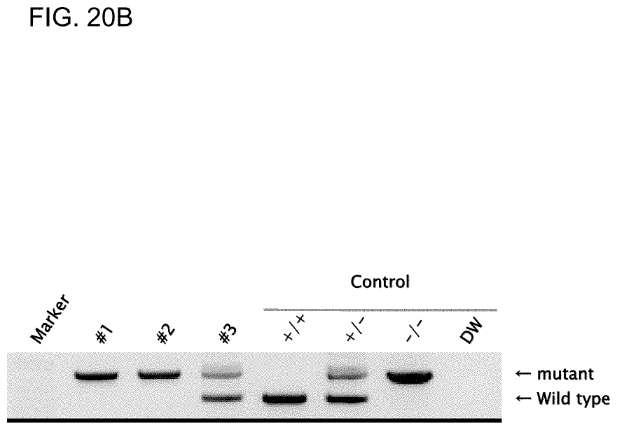

EGFP fluorescence was observed under a fluorescent stereoscopic microscope, where it turned out that litter numbers #1, #2 and #3 are chimeric based on the EGFP expression on the body surface. Upon the Cesarean section, a pancreas with uniform expression of EGFP was observed in #1 and #2. However, the pancreas of #3 exhibited partial expression of EGFP, in a mosaic manner. #4 is a litter-mate as #1-#3, but lacks fluorescence from EGFP, and the pancreas was deficient upon the Cesarean section, and thus it was a non-chimeric Pdx1(−/−) mouse (FIG. 20).

Further, the spleen was removed from these newborn animals, and blood cells prepared therefrom were dyed with a monoclonal antibody against murine or at CD45, and analyzed with a flow cytometer. As a result, in litter numbers #1-#3, rat CD45 positive cells were observed in addition to murine CD45 positive cells, and thus it was confirmed that these are heterologous chimera between mouse and rat containing cells derived from the host mouse and rat iPS cells. Furthermore, almost all cells in the rat CD45 positive cell fractions exhibited fluorescence of EGFP, and thus the rat CD45 positive cell are derived from rat iPS cells marked with EGFP (FIG. 20).

Moreover, in order to confirm that the establishment of blastocyst complementation, i.e. as to whether the deficiency of organs (knocked out) occurred or not, in particular, in order to confirm with an abundance of caution the genotype of the host mice Litter No. #1 to #3, genetic analysis of a single cell, wherein the single cell was from the murine CD45 positive cells collected from spleen samples, for which flow cytometry was conducted as mentioned above, and genomic DNA was extracted therefrom, which were used for genotype judgment.

The primers used for genotype judgment are as follows:

Forward Primer for detection of cell derived from injected embryo (mutant and wild type):

(SEQ ID NO: 9)
ATT GAG ATG AGA ACC GGC ATG

Reverse Primer for detection of cell derived from injected embryo (mutant):

(SEQ ID NO: 10)
TTC AAC ATC ACT GCC AGC TCC

Reverse Primer for detection of cell derived from injected embryo (wild type):

(SEQ ID NO: 11)
TGT GAG CGA GTA ACA ACC

As a result, in #1 and #2, only bands corresponding to mutant type were observed, and in litter No. #3, both bands of mutant type and the wild-type were detected. Therefore, it is understood that the genotype of the host is Pdx1(−/−) in #1 and #2, and in the litter No. #3, it is Pdx1(+/−). From these results, the present inventors have succeeded in the generation of rat pancreas in an individual mouse by applying heterologous blastocyst complementation technology using rat iPS cell as a donor in mice No. #1 and #2, Pdx1(−/−), which should not originally have generated pancreas.

The present invention has been described so far with reference to preferable embodiments, but it should be understood that the scope of the present invention is not restricted by these embodiments but restricted only by the claims. The description in the patents, patent applications and literatures cited herein should be herein incorporated by reference, as described specifically herein.

### INDUSTRIAL APPLICABILITY

According to the method of the present invention, in the body of an individual having deficiency of a certain organ because of its abnormality associated with a lack of the organ development in the development stage, the organ derived from mammalian cells can be formed. In particular, the method of the present invention can be applied even to organs having complicated cellular compositions, such as kidney, pancreas, hair and thymus.

1. A method for producing a target organ in the living body of a non-human mammal having an abnormality associated with a lack of development of the target organ in the development stage, the target organ being derived from an allogeneic and/or xenogeneic mammal that is an individual different from the non-human mammal, the method comprising:
a) preparing a cell derived from the allogeneic and/or xenogeneic mammal;
b) transplanting the cell into a blastocyst stage fertilized egg of the non-human mammal;
c) developing the fertilized egg in the womb of a non-human surrogate parent mammal to obtain a litter; and
d) obtaining the target organ from an individual of the litter.

2. The method according to claim 1, wherein said cell is an embryonic stem cell (ES cell) or an induced pluripotent stem cell (iPS cell).

3. The method according to claim 1, wherein said cell is derived from a mouse.

4. The method according to claim 1, wherein said organ to be produced is selected from a kidney, a pancreas, thymus and hair.

5. The method according to claim 1, wherein said non-human mammal is a mouse.

6. The method according to claim 1, wherein said mouse is a Sall1 knockout mouse, a Pdx-1 knockout mouse, or a nude mouse.

7. The method according to claim 1, wherein said target organ is completely derived from said allogeneic and/or xenogeneic mammal.

8. The method according to claim 1, wherein said cell is derived from a rat.

9. The method according to claim 1, wherein said cell and said non-human mammal are heterologous to each other.

10. The method according to claim 1, wherein said cell is from a rat, and said non-human mammal is from a mouse.

11. A non-human mammal having an abnormality associated with a lack of development of a target organ in the development stage, produced according to a method comprising:
a) preparing a cell derived from an allogeneic and/or xenogeneic mammal that is an individual different from the non-human mammal;
b) transplanting the cell into a blastocyst stage fertilized egg of the non-human mammal; and
c) developing the fertilized egg in the womb of a non-human surrogate parent mammal to obtain a litter.

12. Use of a non-human mammal having an abnormality associated with a lack of development of a target organ in the development stage, for the generation of the target organ.

13. A set for producing a target organ, the set comprising:
A) a non-human mammal having an abnormality associated with a lack of the target organ development in the development stage; and
B) a cell derived from an allogeneic and/or xenogeneic mammal of the same species as the non-human mammal.

14. The set according to claim 13, wherein said cell and said non-human mammal are heterologous to each other.

15. The method according to claim 1, wherein said cell is from a rat, and said non-human mammal is from a mouse.


SEQUENCE LISTING (11 sequences)

SEQ ID NO: 1 - DNA, 20 bp, Artificial Sequence
  primer 1 (wild type allele)
agctaaagctgccagagtgc

SEQ ID NO: 2 - DNA, 20 bp, Artificial Sequence
  primer 2 (common)
caacttgcgattgccataaa

SEQ ID NO: 3 - DNA, 19 bp, Artificial Sequence
  primer 3 (mutant allele)
gcgttggctacccgtgata

SEQ ID NO: 4 - DNA, 19 bp, Artificial Sequence
  nested PCR primer 1 (wild type allele)
agaatgtcgcccgaggttg

SEQ ID NO: 5 - DNA, 19 bp, Artificial Sequence
  nested PCR primer 2 (common)
tacagcaagctaggagcac

SEQ ID NO: 6 - DNA, 19 bp, Artificial Sequence
  nested PCR primer 3 (mutant allele)
aagagcttggcggcgaatg

SEQ ID NO: 7 - DNA, 20 bp, Artificial Sequence
  forward primer in Example 2
caatgatggctccagggtaa

SEQ ID NO: 8 - DNA, 20 bp, Artificial Sequence
  reverse primer in Example 2
tgactttctgtgctcagagg

SEQ ID NO: 9 - DNA, 21 bp, Artificial Sequence
  Forward primer for detection of cell derived from injected embryo (mutant and wild type)
attgagatgagaaccggcatg

SEQ ID NO: 10 - DNA, 21 bp, Artificial Sequence
  Reverse Primer for detection of cell derived from injected embryo (mutant)
ttcaacatcactgccagctcc

SEQ ID NO: 11 - DNA, 18 bp, Artificial Sequence
  Reverse primer for detection of cell derived from injected embryo (wild type)
tgtgagcgagtaacaacc